(19) United States
(12) Patent Application Publication
(10) Pub. No.: US 2018/0155336 A1
(43) Pub. Date: Jun. 7, 2018

(54) PROCESS FOR THE PREPARATION OF 2-{4-[2-({[2-(4-CHLOROPHENYL)-1,3-THIAZOL-4-YL]METHYL}SULFANYL)-3,5-DICYAN0-6-(PYRROLIDIN-1-YL)PYRIDIN-4-YL]PHENOXY}ETHYL-L-ALANYL-L-ALANINATE MONOHYDROCHLORIDE

(71) Applicant: BAYER PHARMA AKTIENGESELLSCHAFT, Berlin (DE)

(72) Inventors: Franz-Josef MAIS, Düsseldorf (DE); Werner HEILMAN, Wuppertal (DE); Britta OLENIK, Bottrop (DE); Birgit KEIL, Düsseldorf (DE); Guido BECKER, Krefeld (DE); Daniel MEIBOM, Wuppertal (DE); Thomas KUHLMANN, Köln (DE)

(73) Assignee: BAYER PHARMA AKTIENGESELLSCHAFT, Berlin (DE)

(21) Appl. No.: 15/568,575

(22) PCT Filed: May 2, 2016
(86) PCT No.: PCT/EP2016/059779

§ 371 Date: Oct. 23, 2017

(30) Foreign Application Priority Data
May 6, 2015 (EP) 15166606.2
Oct. 29, 2015 (EP) 15192030.3

(51) Int. Cl. C07D 417/12

(52) U.S. Cl. CPC C07D 417/12

(57) ABSTRACT

The present application relates to a novel and improved process for preparing the compound 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate monohydrochloride of the formula (I)

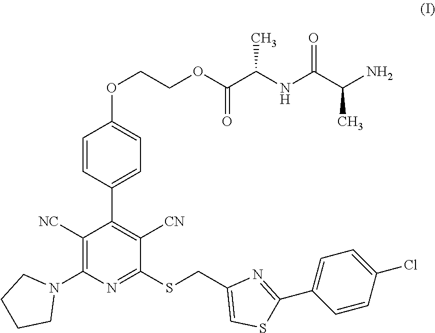 [SMILES: C[C@H](N)C(=O)N[C@@H](C)C(=O)OCCOc1ccc(-c2c(C#N)c(SCc3csc(-c4ccc(Cl)cc4)n3)nc(N3CCCC3)c2C#N)cc1]

to novel precursors for its preparation, and to the preparation and use of crystal modification I of 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}-ethyl-L-alanyl L-alaninate monohydrochloride of the formula (I).

The present application relates to a novel and improved process for preparing the compound 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate monohydrochloride of the formula (I)

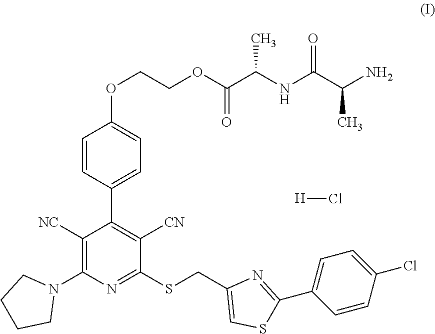 [SMILES: C[C@H](N)C(=O)N[C@@H](C)C(=O)OCCOc1ccc(-c2c(C#N)c(SCc3csc(-c4ccc(Cl)cc4)n3)nc(N3CCCC3)c2C#N)cc1 ; Cl]

to novel precursors for its preparation, and to the preparation and use of crystal modification I of 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate monohydrochloride of the formula (I).

The compound of the formula (I) acts as a partial adenosine A1 receptor agonist and can be used as an agent for the prophylaxis and/or treatment of cardiovascular disorders such as, for example, worsening chronic heart failure, angina pectoris and ischaemic injury during acute coronary syndrome.

2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate hydrochloride and its preparation are described in WO 2010/086101 (see Example 44 therein). The process carried out in Example 44 has the disadvantage that a solid is obtained whose HCl content is stoichiometrically not clearly defined. When Example 44 was emulated (see Example 10 below), the material obtained was amorphous with small amounts of crystals. The HCl content in this solid was about 1.7 mol of HCl per mol of the heterocyclic parent substance. An amorphous material having a stoichiometric composition which is not exactly defined is unsuitable for use as an active compound.

Accordingly, it was an object to provide a process for preparing an exactly defined monohydrochloride of the formula (I) in reproducible crystalline form.

A further disadvantage of the research scale synthesis described therein is the fact that not all steps of this synthesis are suitable for carrying out the processes on a large scale, since many steps proceed at very high dilution, with very high excesses of reagents and therefore afford a relatively low overall yield. Furthermore, many intermediate chromatographic purifications are necessary, which are technically generally very laborious and entail a high consumption of solvents, are costly and are therefore to be avoided if possible. Some steps cannot be realized owing to safety and process engineering restrictions, for example the use of 1-hydroxybenzotriazole (HOBt) as reagent or the use of diethyl ether as solvent.

Accordingly, there existed a need, therefore, for an industrially practicable synthesis, which affords the compound of the formula (I) in a reproducible manner in high overall yield, low production costs, high purity and a pharmaceutically useful crystal form and meets all regulatory requirements, in order to provide clinical trials with active compound and to be used for later regulatory submission.

The present invention provides a process which affords the material of the formula (I) in an exactly defined stoichiometric form as a crystalline product. Furthermore, a very efficient synthesis has been found which allows the requirements mentioned above to be met.

WO 2010/086101 discloses the research scale synthesis of the compound 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate hydrochloride. Starting with 2-amino-6-({[2-(4-chlorophenyl)-1,3-thiazol-4-yl]methyl}sulphanyl)-4-[4-(2-hydroxyethoxy)phenyl]pyridine-3,5-dicarbonitrile (II), also known under the INN capadenoson, the target compound is prepared by the process of the prior art in 6 steps in a yield of 43% of theory. In WO 2010/086101, the target compound is obtained as a solid; however, a defined crystallization process of the end product to set the exact monohydrochloride stoichiometry and to prepare a pharmaceutically useful crystal form or to set the polymorphism has hitherto not been described.

The following scheme 1 shows the known process for preparing the compound of the formula (I).

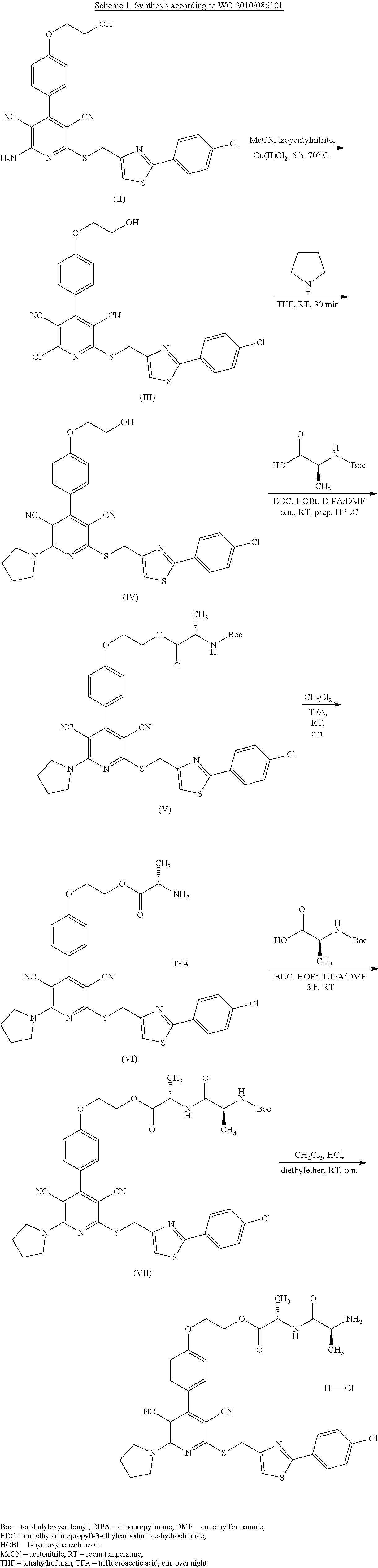

The process proceeds over 6 steps, the step to product (V) being purified chromatographically. For coupling the amino acid building blocks for constructing the side chain, a reagent used is 1-hydroxybenzotriazole (HOBt) which, for safety reasons (risk of explosion) cannot be employed when working on a large scale. Owing to its low flashpoint, the large-scale use of diethyl ether as solvent is likewise not possible without laborious additional safety devices. Furthermore, it is particularly disadvantageous that, using the method described in WO 2010/086101 (Example 44), the target compound is not obtained in a pharmaceutically useful defined crystal form but as an amorphous material not having an exactly defined composition with respect to the HCl content.

Scheme 2 illustrates the novel process according to the invention which affords the compound of the formula (I) in exactly defined stoichiometric form as a crystalline product in 5 steps in a total yield of 55.3% of theory without the need for chromatographic purification.

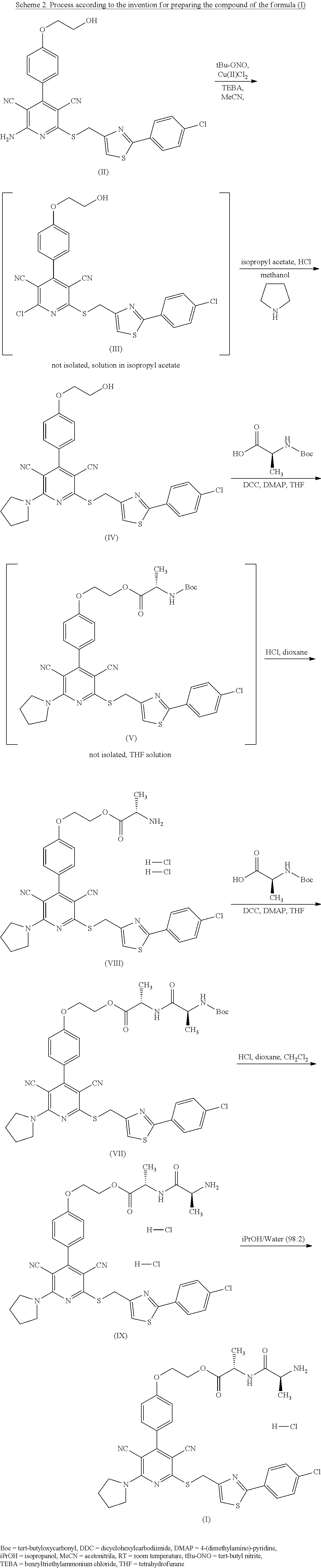

The chloromercaptothiazolopyridine (III) and the protected Boc-alaninate (V) are not isolated but directly reacted further in solution. In one variant, the process can be carried out by also not isolating the protected Boc-dialaninate (VII), but directly reacting further in solution. In this case, the whole process consists only of 4 isolated steps (with a total yield of about 75%) instead of 6 steps in the prior art (with a total yield of about 43%). Scheme 3 shows the process according to the invention taking into account the isolated stages.

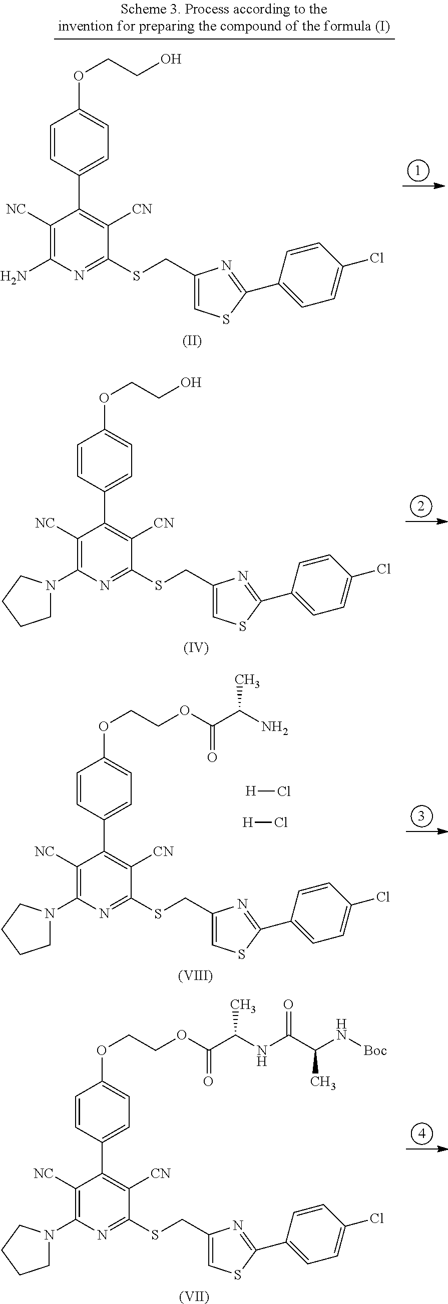

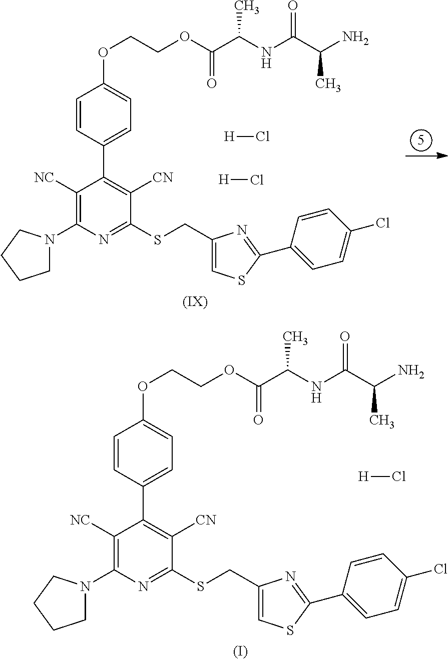

An important advantage of the process according to the invention is the provision of the compound of the formula (I) in exactly defined stoichiometric form as a crystalline product suitable for use as a pharmaceutically active compound.

The individual steps of the process according to the invention for preparing the compound of the formula (I) according to Scheme 3 are discussed below. Alternatives characterized by isolation of the compound of the formula (III) or by the non-isolation of the compound of the formula (VII) are also discussed.

The starting material of the formula (II) is described in WO 03/053441. This compound of the formula (II) is obtained by reacting 2-[4-(2-hydroxyethoxy)benzylidene]malononitrile (XI) with cyanothioacetamide (XII) and 4-(chloromethyl)-2-(4-chlorophenyl)-1,3-thiazole (XIV). Here, the substituted malononitrile (XI) is obtained by reaction of 4-(2-hydroxyethoxy)benzaldehyde (X) with malononitrile. The phenylthiazole (XIV) is obtained by reacting 4-chlorothiobenzamide (XIII) with 1,3-dichloroacetone. This synthesis is summarized in Scheme 4.

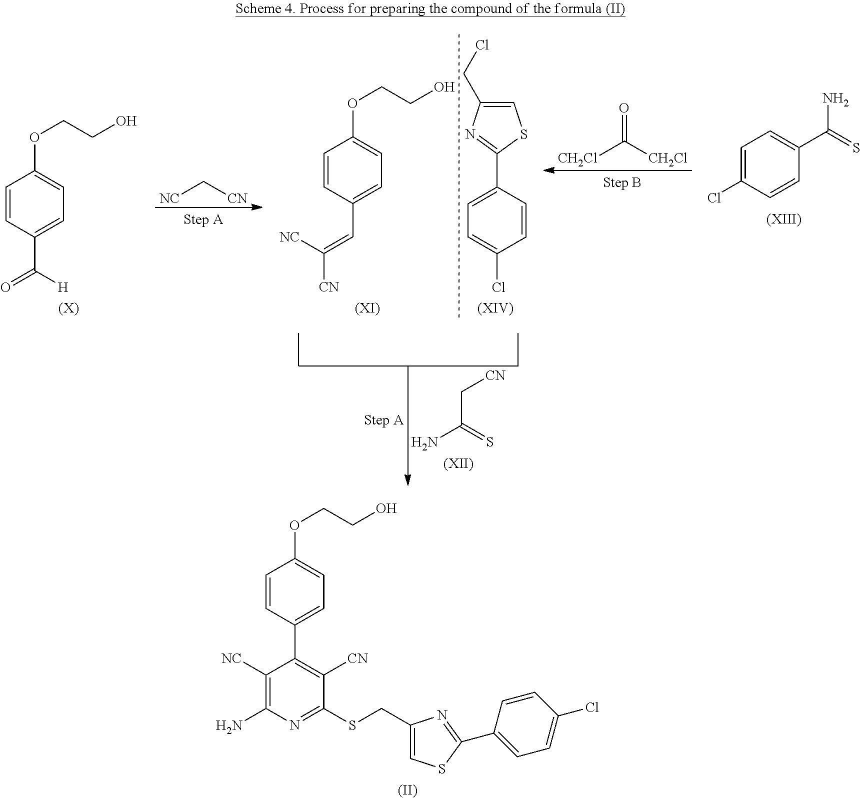

A particular disadvantage of this process is that the reaction of the substituted malononitrile (XI) with the chloromethylchlorophenylthiazole (XIV) and the cyanothioacetamide (XII) (step C) is carried out as a one-pot reaction by adding all three reactants directly in succession. On a large scale, this leads to relatively low yields (68%), with a relatively poor product quality (content about 95%).

According to the invention, in step C during the preparation of the compound of the formula (II), the base/solvent mixture used is now triethylamine/methanol instead of tributylamine/methanol. Additionally, the process is carried out as an advantageous one-pot reaction in a two-step procedure adapted to the chemical mechanism. First, the reactants substituted malononitrile (XI) and cyanothioacetamide (XII) are allowed to react in the presence of triethylamine to give an intermediate. Chloromethylchlorophenylthiazole (XIV) is then added, and the target molecule (II) is formed at slightly elevated temperature (40-50° C.). In this manner, it is possible to achieve a 5% increase in yield (see Example 1).

Hereinbelow, numbering of steps 1 to 5 is based on the preparation in Scheme 3.

Step 1

Step 1 of the process according to the invention was optimized in that the chloromercaptothiazolopyridine (III) is not isolated as intermediate but directly reacted further in solution.

First, 2-amino-6-({[2-(4-chlorophenyl)-1,3-thiazol-4-yl]methyl}sulphanyl)-4-[4-(2-hydroxyethoxy)phenyl]pyridine-3,5-dicarbonitrile (II) is reacted in a solvent such as acetonitrile (alternatively, it is possible to use: proprionitrile, butyronitrile, isobutyronitrile; preference is given to using acetonitrile) in the presence of a phase transfer catalyst such as benzyltriethylammonium chloride (alternatively, it is possible to use: tetrabutylammonium chloride, trimethylbenzylammonium chloride, tributylbenzylammonium chloride; preference is given to using benzyltriethylammonium chloride), in the presence of an oxidizing agent such as tert-butyl nitrite (alternatively, it is possible to use: isoamyl nitrite or isopentyl nitrite, butyl nitrite, isobutyl nitrite; particular preference is given to using tert-butyl nitrite, isoamyl nitrite or isopentyl nitrite) and in the presence of copper(II) chloride. After stirring e.g. in isopropyl acetate and dilute hydrochloric acid, the intermediate chloromercaptothiazolopyridine (III) is obtained in the organic phase.

During the further course of the synthesis, the isopropyl acetate solution of the compound of the formula (III) is diluted, for example, with methanol (alternatively, it is possible to use: short-chain aliphatic alcohols such as methanol, ethanol, propanol, isopropanol; preference is given to using methanol) and reacted with pyrrolidine. The compound of the formula (IV) is obtained in crystalline form.

In the first partial step, typically 1.5 to 4 mol, preferably 1.8 to 3 mol, particularly preferably 2 mol of phase transfer catalyst, 1.5 to 4 mol, preferably 1.8 to 3 mol, particularly preferably 2 mol of the nitrite and 2 to 5 mol, preferably 2.5 to 4 mol, particularly preferably 3 mol of copper(II) chloride are employed per mole of the compound of the formula (II).

First, the starting materials phase transfer catalyst, solvent and compound of the formula (II) are combined at room temperature. After addition of copper(II) chloride, the mixture is heated to 40° C. to the boiling point of the solvent, preferably 40 to 60° C., particularly preferably 50° C., and, after the addition of the nitrite (e.g. tert-butyl nitrite), stirred for another 2 to 12 h, preferably 3 to 6 h, particularly preferably 4 h, at 50° C. When the reaction has ended, the mixture is cooled to room temperature (20 to 30° C.).

Work-up takes place by stirring with isopropyl acetate (alternatively, it is possible to use: ethyl acetate, propyl acetate, butyl acetate, ethyl propionate; preference is given to using isopropyl acetate) and dilute hydrochloric acid (5 to 20% in water, preferably 5 to 10% in water, particularly preferably 7% in water) and subsequent clarification.

In the second partial step, typically 3 to 10 mol, preferably 3.5 to 7 mol, particularly preferably 4 mol, of pyrrolidine are employed per mole of chloromercaptothiazolopyridine (III).

The addition of pyrrolidine is carried out with slight cooling such that the temperature does not exceed 35° C., preferably 30° C., particularly preferably 25° C. The mixture is then first stirred for 1 to 5 h, preferably 1.5 to 3 h, particularly preferably 2 h, at this temperature and then overnight (10 to 24 h, preferably 15 to 20 h) at 50° C. to reflux of the solvent, preferably at 60 to 70° C., or particularly preferably in methanol at reflux (about 66° C.).

Work-up takes place by cooling to 0 to 10° C., preferably about 5° C. The product of the formula (IV) is filtered, washed and dried.

Using this reaction regime, the yield is about 84% over the two partial steps, whereas the synthesis known from the literature only affords a yield of about 56% over the two steps.

Alternative with Isolation of the Chloromercaptothiazolopyridine (III)

As an alternative to this one-pot reaction, the reaction can also be carried out with isolation of the chloromercaptothiazolopyridine of the formula (III). The reaction regime according to the invention increases the yield in both partial reactions, from about 69% to about 79% in the first partial step and from about 81% to about 91% in the second partial step.

As in the one-pot process, first, 2-amino-6-({[2-(4-chlorophenyl)-1,3-thiazol-4-yl]methyl}sulphanyl)-4-[4-(2-hydroxyethoxy)phenyl]pyridine-3,5-dicarbonitrile (II) is reacted in a solvent such as acetonitrile (alternatively, it is possible to use: proprionitrile, butyronitrile, isobutyronitrile; preference is given to using acetonitrile) in the presence of a phase transfer catalyst such as benzyltriethylammonium chloride (alternatively, it is possible to use: tetrabutylammonium chloride, trimethylbenzylammonium chloride, tributylbenzylammonium chloride; preference is given to using benzyltriethylammonium chloride), in the presence of an oxidizing agent such as tert-butyl nitrite (alternatively, it is possible to use: isoamyl nitrite or isopentyl nitrite, butyl nitrite, isobutyl nitrite; particular preference is given to using tert-butyl nitrite, isoamyl nitrite or isopentyl nitrite) and in the presence of copper(II) chloride. After stirring in ethyl acetate and water, the intermediate chloromercaptothiazolopyridine (III) is obtained in the organic phase. Distillation and recrystallization from methanol gave the compound of the formula (III).

In this reaction regime, 1.5 to 4 mol, preferably 1.8 to 3 mol, particularly preferably 2 mol of phase transfer catalyst, 1.5 to 4 mol, preferably 1.8 to 3 mol, particularly preferably 2 mol of the nitrite and 2 to 5 mol, preferably 2.5 to 4 mol, particularly preferably about 3 mol of copper(II) chloride are employed per mole of the compound of the formula (II).

First, the starting materials phase transfer catalyst, solvent and compound of the formula (II) are combined at room temperature. After addition of copper(II) chloride, the mixture is heated to 40° C. to the boiling point of the solvent, preferably 40 to 60° C., particularly preferably 50° C., and, after the addition of the nitrite (e.g. tert-butyl nitrite), stirred for another 2 to 12 h, preferably 3 to 6 h, particularly preferably 4 h, at 50° C. When the reaction has ended, the mixture is cooled to room temperature (20 to 30° C.).

Work-up takes place by stirring with ethyl acetate (alternatively, it is possible to use: isopropyl acetate, propyl acetate, butyl acetate, ethyl propionate; particular preference is given to using ethyl acetate or isopropyl acetate, particularly preferably ethyl acetate) and water. The organic phases are then distilled. The residue gives the compound of the formula (III) after recrystallization from methanol (alternatively, it is possible to use: short-chain aliphatic alcohols such as methanol, ethanol, propanol, isopropanol; preference is given to using methanol).

During the further course of the synthesis, the isolated compound of the formula (III) is suspended, for example, in methanol (alternatively, it is possible to use: short-chain aliphatic alcohols such as methanol, ethanol, propanol, isopropanol; preference is given to using methanol) and reacted with pyrrolidine. The compound of the formula (IV) is obtained in crystalline form.

Typically, 3 to 10 mol, preferably 3.5 to 7.5 mol, of pyrrolidine are employed per mole of isolated chloromercaptothiazolopyridine (III).

The addition of pyrrolidine is carried out with slight cooling such that the temperature does not exceed 35° C., preferably 30° C., particularly preferably 25° C. The mixture is then first stirred for 1 to 5 h, preferably 1.5 to 3 h, particularly preferably 2 h, at this temperature and then overnight (10 to 24 h, preferably 15 to 20 h) at 50° C. to reflux of the solvent, preferably at 60 to 70° C., or particularly preferably in methanol at reflux (about 65 to 66° C.).

Work-up takes place by cooling to 0 to 10° C., preferably about 5° C. The product of the formula (IV) is filtered, washed and dried.

Step 2

Step 2 of the process according to the invention was optimized in that the protected Boc-alaninate (V) is not isolated as intermediate but directly reacted further in solution. For carrying out this step, conditions particularly suitable for large-scale synthesis are chosen. In this amide coupling with subsequent removal of the protective group, the chosen reaction parameters gave quantitative yields of alaninate dihydrochloride of the formula (VIII).

First, the compound of the formula (IV) and Boc-L-alanine are initially charged in a solvent such as tetrahydrofuran (alternatively, it is possible to use: dioxane, methyltetrahydrofuran; preference is given to using tetrahydrofuran) and the mixture is then stirred in the presence of dicyclohexylcarbodiimide (DCC) in combination with 4-(dimethylamino)pyridine.

In this partial step, typically 1 to 3 mol, preferably 1.2 to 2 mol, particularly preferably 1.3 to 1.5 mol of Boc-L-alanine, 0.2 mol to 1.5 mol, preferably 0.4 to 1 mol, particularly preferably about 0,5 mol, of 4-(dimethylamino)pyridine and 1 to 3 mol, preferably 1.2 to 2 mol, particularly preferably about 1.5 mol, of DCC are employed per mole of the compound of the formula (IV).

The starting materials first combined at 10 to 35° C., preferably 20 to 30° C., preferably room temperature, and then stirred at this temperature overnight (10 to 24 h, preferably 15 to 20 h).

Work-up takes place by removing solid constituents by filtration with suction and rinsing with the solvent employed, such as tetrahydrofuran. The filtrate is then concentrated to a fraction of 15 to 30% of the total amount, preferably about 20% of the total amount.

In the second partial step of this step 2, typically 10 to 20 mol, preferably 11 to 15 mol, particularly preferably about 12 mol of hydrochloric acid (in the form of a 4 M solution in dioxane) are employed per mole of non-isolated protected Boc-alaninate (V).

The addition of the hydrochloric acid is carried out with slight cooling such that the temperature does not exceed 15 to 30° C., preferably 20-25° C. The mixture is then stirred for 5 to 20 h, preferably 10 to 15 h, particularly preferably about 12 h, at 15 to 30° C., preferably 20-25° C. Work-up takes place by filtration, washing and drying of the precipitated solid.

The yield is quantitative over the two partial steps. The formally calculated yield of more than 100% is due to reagent and solvent residues and to dimethylaminopyridine hydrochloride, which is present in the isolated product. These impurities do not reduce the quality of the alaninate dihydrochloride intermediate (VIII), since all reagents are used again in the next step and the subsequent stages Boc-dialaninate (VII) and dialaninate dihydrochloride (IX) are purified completely.

Step 3

Step 3 of the process according to the invention was optimized such that isolation and purification of the protected Boc-dialaninate of the formula (VII) are carried out without chromatographic work-up.

To this end, first the compound of the formula (VIII) and 4-(dimethylamino)pyridine (DMAP) are initially charged in a solvent such as tetrahydrofuran (THF) (alternatively, it is possible to use: dioxane, methyltetrahydrofuran; preference is given to using tetrahydrofuran). After addition of Boc-L-alanine and dicyclohexylcarbodiimide (DCC), the reaction mixture is stirred.

Typically, 1 to 3 mol, preferably 1.1 to 2 mol, particularly preferably 1.2 to 1.5 mol of Boc-L-alanine, 1 to 4 mol, preferably 1.5 to 3 mol, particularly preferably 2 mol, of 4-(dimethylamino)pyridine and 1 to 3 mol, preferably 1.1 to 2 mol, particularly preferably about 1.2 mol, of DCC are employed per mole of the compound of the formula (VIII).

The starting materials compound of the formula (VIII), DMAP and THF are first combined at 10 to 35° C., preferably 20 to 30° C., preferably 20 to 25° C., Boc-L-alanine is added and, after addition of DCC, the mixture is stirred at this temperature for 2 to 12 h, preferably 6 to 8 h, particularly preferably about 4 h.

Work-up takes place by removal of solid constituents by filtration with suction and washing with a 10 to 20% strength, preferably about 15% strength, solution of ammonium chloride in water. Repeatedly, dioxane is then added to the organic phase and distilled off. Following crystallization by addition of diisopropyl ether, the Boc-protected dialaninate (VII) is obtained in crystalline form.

Step 4

For carrying out step 4, the protected Boc-dialaninate (VII) is first dissolved in hot dichloromethane and filtered, and excess dichloromethane is distilled off. The bottom is diluted with diisopropyl ether, and 10 to 20 mol, preferably 11 to 15 mol, particularly preferably about 12 mol, of hydrochloric acid are added per mole of compound (VII) (in the form of a 4 M solution in dioxane).

The addition of the hydrochloric acid is carried out such that the internal temperature does not exceed 15 to 30° C., preferably 20 to 25° C. The mixture is then stirred at this temperature for 5 to 24 h, preferably 10 to 20 h, particularly preferably about 12 to 16 h.

Work-up takes place by filtration of the precipitated solid.

The total yield over step 3 and step 4 is very high (96.9%), whereas the synthesis known from the literature affords only 88% yield over 2 steps. Here, the starting material used is the alaninate trifluoroacetate of the formula (VI) instead of the alaninate dihydrochloride (VIII).

Alternative to Step 3 and Step 4 Without Intermediate Isolation of the Boc-Dialaninate (VII)

In an alternative configuration, step 3 of the process according to the invention was optimized in that the protected Boc-dialaninate (VII) is not isolated as intermediate but directly reacted further in solution. For carrying out this step, conditions particularly suitable for large-scale synthesis are chosen. In this amide coupling with subsequent removal of the protective group, the chosen reaction parameters gave almost quantitative yields of dialaninate dihydrochloride of the formula (IX).

First, the compound of the formula (VIII) and 4-(dimethylamino)pyridine (DMAP) are initially charged in a solvent such as tetrahydrofuran (THF) (alternatively, it is possible to use: dioxane, methyltetrahydrofuran; preference is given to using tetrahydrofuran). After addition of Boc-L-alanine and dicyclohexylcarbodiimide (DCC), the reaction mixture is stirred.

Typically, 1 to 3 mol, preferably 1.1 to 2 mol, particularly preferably 1.2 to 1.5 mol of Boc-L-alanine, 1 to 4 mol, preferably 1.5 to 3 mol, particularly preferably 2 mol, of 4-(dimethylamino)pyridine and 1 to 3 mol, preferably 1.1 to 2 mol, particularly preferably about 1.2 mol, of DCC are employed per mole of the compound of the formula (VIII).

The starting materials compound of the formula (VIII), DMAP and THF are first combined at 10 to 35° C., preferably 20 to 30° C., preferably 20 to 25° C., Boc-L-alanine is added and, after addition of DCC, the mixture is stirred at this temperature for 2 to 12 h, preferably 6 to 8 h, particularly preferably about 4 h.

Work-up takes place by removal of solid constituents by filtration with suction and washing with a 10 to 20% strength, preferably about 15% strength, solution of ammonium chloride in water. Repeatedly, dioxane is then added to the organic phase and distilled off. The protected Boc-dialaninate (VII) as a solution in dioxane is then used in the next partial step.

In the second partial step of this step 3, typically 10 to 20 mol, preferably 11 to 15 mol, particularly preferably about 12 mol of hydrochloric acid [per mole of the compound (VII)] (in the form of a 4 M solution in dioxane) are employed per mole of non-isolated protected Boc-dialaninate (VII).

The addition of the hydrochloric acid is carried out such that the internal temperature does not exceed 15 to 30° C., preferably 20 to 25° C. The mixture is then stirred at this temperature for 5 to 24 h, preferably 10 to 20 h, particularly preferably about 12 to 16 h.

Work-up takes place by filtration of the precipitated solid.

The yield is almost quantitative (98%) over the two partial steps, whereas the synthesis known from the literature only affords a yield of 88% over 2 steps.

Step 5

Step 5 of the process according to the invention is an entirely new step which affords the hydrochloride of the formula (I) from the dialaninate dihydrochloride (IX) under very particular conditions. Here, the stoichiometric content of HCl is adjusted exactly to the monohydrochloride stage and the product of the formula (I) is obtained in the crystalline modification I according to the invention.

In step 5, the dialaninate dihydrochloride (IX) is stirred in a 10- to 25-fold, preferably 12- to 20-fold, particularly preferably an about 15-fold excess of an alcohol/water mixture (proportion of water 0.5 to 5% by volume, preferably 1 to 3% by volume, particularly preferably about 2% by volume, alcohol: isopropanol or n-propanol, preferably isopropanol), filtered off, washed with the alcohol and dried. Alternatively, it is also possible to use only the alcohol, without addition of water.

The isopropanol/water mixture is added to the dialaninate dihydrochloride (IX) at 10 to 35° C., preferably 15 to 30° C., particularly preferably at 23 to 28° C., and the mixture is then stirred at this temperature for 6 to 96 h, preferably 12 to 84 h, particularly preferably 18 to 72 h.

Work-up is carried out by filtration, repeated washing with the alcohol and drying. If stirring is carried out in the preferred solvent isopropanol, filtration and washing initially yields an isopropanol solvate of low thermal stability which, by subsequent drying, is converted completely into the crystalline form of modification (I).

Since the compound of the formula (I) has been developed in the form of a tablet, there exists a high demand that the isolated compound of the formula (I) is isolated in a reproducible manner in an exactly defined HCl stoichiometry and in a defined crystalline form such that reproducible active compound contents in tablet production and a reproducible bioavailability can be ensured. Surprisingly, it has been found that the compound of the formula (I) can be crystallized from isopropanol or n-propanol/water (98:2), giving, in a reproducible manner, the crystalline modification I, which has a melting point of 156 to 166° C.

For further purification, the isolated moist product can, prior to drying, be stirred again with the isopropanol or n-propanol/water mixture (proportion of water 0.5 to 5% by volume, preferably 1 to 3% by volume, particularly preferably about 2% by volume), in a 10- to 25-fold, preferably 12- to 20-fold, particularly preferably in an about 15-fold excess, for 1 to 36 h, preferably 12 to 24 h. The mixture is subsequently filtered off and the product is washed and dried under reduced pressure.

The present invention provides the compound of the formula (I) in crystalline form of modification I, characterized in that the X-ray diffractogram of the compound has peak maxima of the 2 theta angle at 6.5, 8.7 and 24.3.

The present invention provides the compound of the formula (I) in crystalline form of modification I, characterized in that the X-ray diffractogram of the compound has peak maxima of the 2 theta angle at 6.5, 8.7, 18.3, 19.9, 20.7, 23.5 and 24.3.

The present invention further provides the compound of the formula (I) in crystalline form of modification I, characterized in that the Raman spectrum of the compound has band maxima at 2907, 1004 and 598 cm⁻¹.

The present invention further provides the compound of the formula (I) in crystalline form of modification I, characterized in that the Raman spectrum of the compound has band maxima at 2907, 1539, 1515, 1182, 1004 and 598 cm⁻¹.

The present invention further provides the compound of the formula (I) in crystalline form of modification I, characterized in that the Raman spectrum of the compound has band maxima at 2907, 1539, 1515, 1394, 1245, 1182, 1004 and 598 cm⁻¹.

The present invention furthermore provides a process for preparing the compound of the formula (I) in crystalline form of modification I, characterized in that the dichloride of the formula (IX) is stirred for several hours in isopropanol or n-propanol/water (98:2 v/v), then filtered, washed and dried under reduced pressure.

Preferred solvent for the process for preparing the compound of the formula (I) in crystalline form of modification I is isopropanol/water (98:2 v/v).

A preferred temperature range for the process for preparing the compound of the formula (I) in crystalline form of modification I is from 20 to 30° C.

The present invention further provides the compound of the formula (I) in crystalline form of modification I as described above for treatment of disorders.

The present invention further provides a medicament comprising a compound of the formula (I) in crystalline form of modification I as described above and no major proportions of any other form of the compound of the formula (I) than the crystalline form of modification I as described above. Medicament comprising a compound of the formula (I) in crystalline form of modification I as described above in more than 90 percent by weight based on the total amount of the compound of the formula (I) present in crystalline form of modification I as described above.

The present invention further provides for the use of the compound of the formula (I) in crystalline form of modification I as described above for production of a medicament for treatment of cardiovascular disorders.

The present invention further provides the method for treatment of cardiovascular disorders by administering an effective amount of a compound of the formula (I) in crystalline form of modification I as described above.

The present invention further provides a process for preparing the compound of the formula (I) in crystalline form of modification I, characterized in that the dialaninate dihydrochloride (IX) is stirred in an excess of isopropanol/water mixture (98:2 v/v) at room temperature and the compound of the formula (I) in crystalline modification I is obtained after drying.

The present invention further provides a process for preparing compound (I), characterized in that
[A] in step 1 in a one-pot reaction the compound of the formula (II)

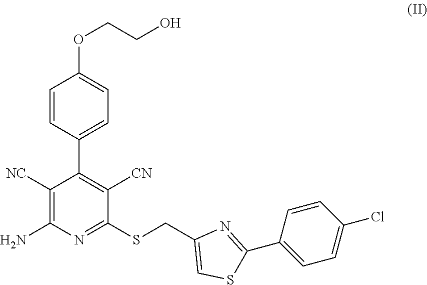 [SMILES: N#Cc1c(N)nc(SCc2csc(-c3ccc(Cl)cc3)n2)c(C#N)c1-c1ccc(OCCO)cc1]

is reacted in the presence of a solvent, a phase transfer catalyst, a nitrite and copper(II) chloride and the intermediate of the formula (III) obtained

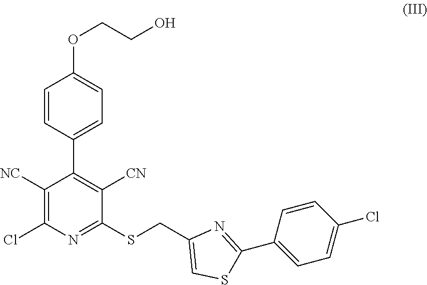 [SMILES: N#Cc1c(Cl)nc(SCc2csc(-c3ccc(Cl)cc3)n2)c(C#N)c1-c1ccc(OCCO)cc1]

is reacted without isolation, i.e. in solution, with pyrrolidine to give the compound of the formula (IV)

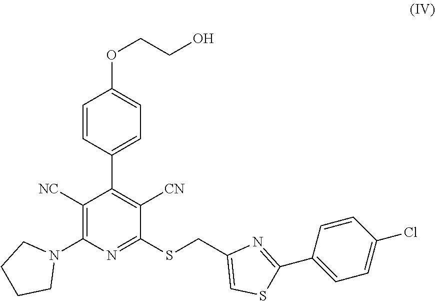 [SMILES: N#Cc1c(SCc2csc(-c3ccc(Cl)cc3)n2)nc(N2CCCC2)c(C#N)c1-c1ccc(OCCO)cc1]

and
[B] the compound of the formula (IV) obtained in this manner is, in a step 2, converted in a one-pot reaction with Boc-L-alanine in the presence of the condensing agent dicyclohexylcarbodiimide (DCC) in combination with 4-(dimethylamino)pyridine and in the presence of a solvent into the protected Boc-alaninate (V)

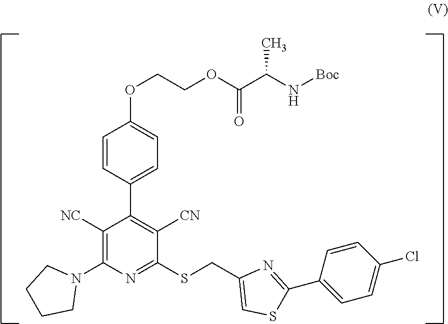

which is, without isolation, i.e. in solution, converted with hydrochloric acid into the alaninate dihydrochloride of the formula (VIII)

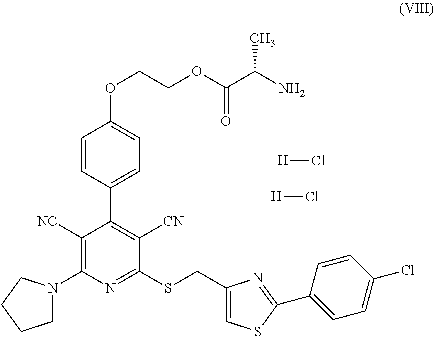

and
[C] the compound of the formula (VIII) obtained in this manner is, in a step 3, in combination with 4-(dimethylamino)pyridine and in the presence of a solvent, reacted with Boc-L-alanine in the presence of the condensing agent dicyclohexylcarbodiimide (DCC) to give the protected Boc-dialaninate (VII)

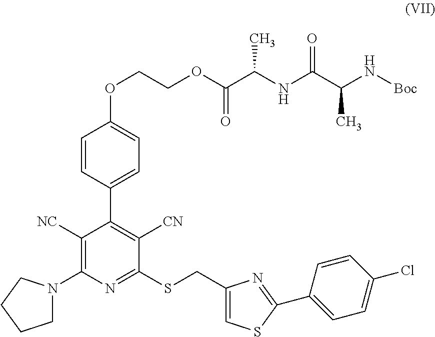 [SMILES: C[C@H](NC(=O)OC(C)(C)C)C(=O)N[C@@H](C)C(=O)OCCOc1ccc(-c2c(C#N)c(SCc3csc(-c4ccc(Cl)cc4)n3)nc(N3CCCC3)c2C#N)cc1]

and
[D] the compound of the formula (VII) obtained in this manner is, in a step 4, in the presence of a solvent, reacted with hydrochloric acid to give the dialaninate dihydrochloride of the formula (IX)

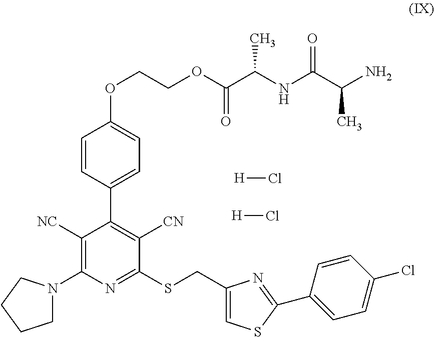

and
[E] the compound of the formula (IX) obtained in this manner is, in a step 5, stirred in an excess of isopropanol or n-propanol/water mixture (98:2) at room temperature, giving, after drying, the compound of the formula (I) in crystalline modification I.

The present invention furthermore provides a process for preparing compound (I) in crystalline modification I, characterized in that in step 1 the intermediate of the formula (III) is isolated, i.e. obtained as a solid, prior to being reacted further.

The present invention furthermore provides a process for preparing compound (I) in crystalline modification I, characterized in that in step 2 the protected Boc-alaninate of the formula (V) is isolated, i.e. obtained as a solid, prior to being reacted further.

The present invention furthermore provides a process for preparing compound (I) in crystalline modification I, characterized in that the protected Boc-dialaninate of the formula (VII) obtained in step 3 is not isolated but directly reacted further.

The present invention further provides a process for preparing compound (I), characterized in that in step 1 the phase transfer catalyst used is benzyltriethylammonium chloride.

The present invention further provides a process for preparing compound (I), characterized in that in step 1 the oxidizing agent used is tert-butyl nitrite.

The present invention further provides a process for preparing compound (I), characterized in that in step 1 the phase transfer catalyst used is benzyltriethylammonium chloride and the oxidizing agent used is tert-butyl nitrite.

The present invention further provides the dialaninate dihydrochloride of the formula (IX)

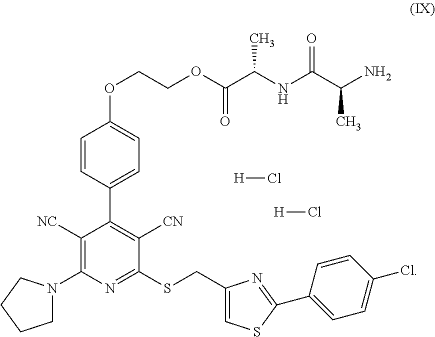

The present invention further provides a process for preparing the dialaninate dihydrochloride of the formula (IX), characterized in that the alaninate dihydrochloride of the formula (VIII) is reacted with Boc-L-alanine in the presence of a condensing agent (e.g. dicyclohexylcarbodiimide) and in the presence of 4-(dimethylamino)pyridine in a solvent (e.g. tetrahydrofuran) and the protected Boc-dialaninate (VII) obtained in this manner is reacted either without isolation in solution or after isolation with hydrochloric acid in a solvent (e.g. dioxane).

The present invention further provides the alaninate dihydrochloride of the formula (VIII)

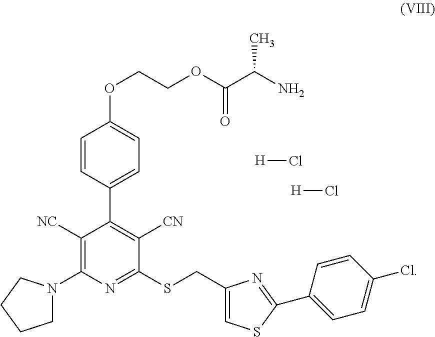

The present invention further provides a process for preparing the alaninate dihydrochloride of the formula (VIII), characterized in that the compound of the formula (IV) is reacted with Boc-L-alanine in the presence of a condensing agent (e.g. dicyclohexylcarbodiimide) and in the presence of 4-(dimethylamino)pyridine in a solvent (e.g. tetrahydrofuran) and the protected Boc-dialaninate (VII) obtained in this manner is reacted either without isolation in solution or after isolation with hydrochloric acid in a solvent (e.g. tetrahydrofuran).

The compound of the formula (I) is generally micronized and formulated in pharmacy. It is found that the compound of the formula (I) in crystalline form of modification I has very good stability properties (even at high atmospheric humidity) and can be stored without any problem for >2 years.

With the novel synthesis according to the invention, it is possible to prepare the compound of the formula (I) in a very efficient manner The process offers considerable advantages compared to the prior art relating to scalability and technical performance. The overall yield is significantly higher compared to published data and excellent purities of the active ingredient are also achieved. The novel process enables the reproducible, economic preparation of the defined compound of the formula (I) in crystalline form of modification I, of which the existence in the prior art has hitherto not been described.

Surprisingly, three further modifications and an isopropanol solvate of the compound of the formula (I) have been found. These can be obtained by initially micronizing the compound of the formula (I) obtained by the process according to the invention, and then treating it as follows:

Drying in a dessicator over phosphorus pentoxide gave modification II having a melting point of 146° C.

Modification III having a transition point of 134° C. was obtained by initially suspending micronized material in acetonitrile, then stirring at room temperature for one week and then allowing to stand at room temperature until the solvent has evaporated.

Modification IV having a melting point of 122° C. was obtained by initially dissolving micronized material in methanol, then allowing to stand at room temperature until the solvent has evaporated.

An isopropanol solvate was obtained by stirring the intermediate dialaninate dihydrochloride of the formula (IX) at room temperature with isopropanol/water (98:2), isolating the solid and air-drying without reducing the pressure, at room temperature.

The compounds according to the invention, the compound of the formula (I) and the compound of the formula (I) in crystalline form of modification I act as partial adenosine A1 receptor agonists and exhibit an unforeseeable, useful spectrum of pharmacological activity. They are therefore suitable for use as medicaments for treatment and/or prophylaxis of disorders in humans and animals.

The compounds according to the invention, on their own or in combination with one or more other active compounds, are suitable for the prevention and/or treatment of various disorders, for example disorders of the cardiovascular system (cardiovascular disorders), for cardioprotection after damage to the heart and of metabolic and kidney disorders.

For the purpose of the present invention, disorders of the cardiovascular system or cardiovascular disorders are to be understood as meaning, for example, the following disorders: hypertension, peripheral and cardiac vascular disorders, coronary heart disease, coronary restenosis such as, for example, restenosis after balloon dilatation of peripheral blood vessels, myocardial infarction, acute coronary syndrome, acute coronary syndrome with ST elevation, acute coronary syndrome without ST elevation, stable and unstable angina pectoris, myocardial insufficiency, Prinzmetal angina, persistent ischaemic dysfunction (“hibernating myocardium”), transient postischemic dysfunction (“stunned myocardium”), heart failure, tachycardias, atrial tachycardia, arrhythmias, atrial and ventricular fibrillation, persistent atrial fibrillation, permanent atrial fibrillation, atrial fibrillation with normal left ventricular function, atrial fibrillation with impaired left ventricular function, Wolff-Parkinson-White syndrome, disturbances of peripheral blood flow, elevated levels of fibrinogen and of low density LDL, and elevated concentrations of plasminogen activator inhibitor 1 (PAI-1), especially coronary heart disease, acute coronary syndrome, angina pectoris, heart failure, myocardial infarction and atrial fibrillation.

For the purpose of the present invention, the term heart failure includes both acute and chronic manifestations of heart failure, as well as more specific or related types of disease, such as acute decompensated heart failure, right heart failure, left heart failure, global failure, ischaemic cardiomyopathy, dilated cardiomyopathy, congenital heart defects, heart valve defects, heart failure associated with heart valve defects, mitral stenosis, mitral insufficiency, aortic stenosis, aortic insufficiency, tricuspid stenosis, tricuspid insufficiency, pulmonary stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, and diastolic and systolic heart failure and acute phases of worsening heart failure.

The compounds according to the invention are furthermore also suitable for reducing the myocard region affected by an infarction, and for the prevention of secondary infarctions.

The compounds according to the invention are furthermore suitable for the prevention and/or treatment of thromboembolic disorders, reperfusion damage following ischaemia, micro- and macrovascular lesions (vasculitis), arterial and venous thromboses, oedemas, ischaemias such as myocardial infarction, stroke and transient ischaemic attacks, for cardioprotection in connection with coronary artery bypass operations (CABG), primary percutaneous transluminal coronary angioplasties (PTCAs), PTCAs after thrombolysis, rescue PTCA, heart transplants and open-heart operations, and for organ protection in connection with transplants, bypass operations, heart catheter examinations and other surgical procedures.

Other areas of indication for which the compounds according to the invention can be employed are, for example, the prevention and/or treatment of disorders of the urogenital tract, for example irritable bladder, erectile dysfunction and female sexual dysfunction, but in addition also the prevention and/or treatment of inflammatory disorders, for example inflammatory dermatoses (psoriasis, acne, eczema, neurodermitis, dermatitis, keratitis, formation of scars, formation of warts, frostbites), of disorders of the central nervous system and neurodegenerative disorders (stroke, Alzheimer's disease, Parkinson's disease, dementia, epilepsy, depression, multiple sclerosis), of states of pain, cancerous diseases (skin cancer, liposarcomas, carcinomas of the gastrointestinal tract, the liver, pancreas, lung, kidney, ureter, prostate and the genital tract), and also of nausea and emesis associated with cancer therapies.

Other areas of indication are, for example, the prevention and/or treatment of inflammatory and immune disorders (Crohn's disease, ulcerative colitis, lupus erythematosus, rheumatoid arthritis) and respiratory disorders, such as, for example, chronic obstructive pulmonary disease (chronic bronchitis, COPD), asthma, pulmonary emphysema, bronchiectases, cystic fibrosis (mucoviscidosis) and pulmonary hypertension, in particular pulmonary arterial hypertension.

Finally, the compounds according to the invention are also suitable for the prevention and/or treatment of diabetes, in particular diabetes mellitus, gestation diabetes, insulin-dependent diabetes and non-insulin-dependent diabetes, of diabetic sequelae such as, for example, retinopathy, nephropathy and neuropathy, of metabolic disorders (metabolic syndrome, hyperglycaemia, gestation diabetes, hyperinsulinaemia, insulin resistance, glucose intolerance, obesity (adipositas)) and also of arteriosclerosis and dyslipidemias (hypercholesterolemia, hypertriglyceridemia, elevated concentrations of postprandial plasma triglycerides, hypoalphalipoproteinemia, combined hyperlipidemias), in particular of diabetes, metabolic syndrome and dyslipidemias.

In addition, the compounds according to the invention can also be used for the treatment and/or prevention of disorders of the thyroid gland (hyperthyreosis), disorders of the pancreas (pancreatitis), fibrosis of the liver, viral disorders (HPV, HCMV, HIV), cachexia, osteoporosis, gout, incontinence, and also for wound healing and angiogenesis.

The present invention further provides for the use of the compounds according to the invention for treatment and/or prevention of disorders, especially the aforementioned disorders.

The present invention further provides for the use of the compounds according to the invention for producing a medicament for the treatment and/or prevention of disorders, in particular the disorders mentioned above.

The present invention further provides a process for treatment and/or prevention of disorders, in particular the disorders mentioned above, using an effective amount of at least one of the compounds according to the invention.

The present invention further provides the compounds according to the invention for use in a method for the treatment and/or prophylaxis of coronary heart disease, acute coronary syndrome, angina pectoris, heart failure, myocardial infarction and atrial fibrillation.

The present invention further provides the compounds according to the invention for methods for the treatment and/or prophylaxis of diabetes, metabolic syndrome and dyslipidemias.

The compounds of the invention can be used alone or, if required, in combination with other active ingredients. The present invention further provides medicaments comprising at least one of the compounds of the invention and one or more further active ingredients, especially for treatment and/or prevention of the aforementioned disorders.

Active compounds suitable for combination are, by way of example and with preference: active compounds which modulate lipid metabolism, antidiabetics, hypotensive agents, perfusion-enhancing and/or antithrombotic agents, antioxidants, chemokine receptor antagonists, p38 kinase inhibitors, NPY agonists, orexin agonists, anorectics, PAF-AH inhibitors, antiphlogistics (COX inhibitors, LTB4-receptor antagonists), analgesics, for example aspirin, antidepressants and other psychopharmaceuticals.

The present invention provides in particular combinations of at least one of the compounds according to the invention and at least one lipid metabolism-modifying active compound, antidiabetic, hypotensive active compound and/or agent having antithrombotic action.

The compounds according to the invention can preferably be combined with one or more
lipid metabolism-modulating active ingredients, by way of example and with preference from the group of the HMG-CoA reductase inhibitors, inhibitors of HMG-CoA reductase expression, squalene synthesis inhibitors, ACAT inhibitors, LDL receptor inductors, cholesterol absorption inhibitors, polymeric bile acid adsorbers, bile acid reabsorption inhibitors, MTP inhibitors, lipase inhibitors, LpL activators, fibrates, niacin, CETP inhibitors, PPAR-α, PPAR-γ and/or PPAR-δ agonists, RXR modulators, FXR modulators, LXR modulators, thyroid hormones and/or thyroid mimetics, ATP citrate lyase inhibitors, Lp(a) antagonists, cannabinoid receptor 1 antagonists, leptin receptor agonists, bombesin receptor agonists, histamine receptor agonists and the antioxidants/radical scavenger;
antidiabetics mentioned in the Rote Liste 2004/II, chapter 12, and also, by way of example and with preference, those from the group of the sulfonylureas, biguanides, meglitinide derivatives, glucosidase inhibitors, inhibitors of dipeptidyl-peptidase IV (DPP-IV inhibitors), oxadiazolidinones, thiazolidinediones, GLP 1 receptor agonists, glucagon antagonists, insulin sensitizers, CCK 1 receptor agonists, leptin receptor agonists, inhibitors of liver enzymes involved in the stimulation of gluconeogenesis and/or glycogenolysis, modulators of glucose uptake and also potassium channel openers, such as, for example, those disclosed in WO 97/26265 and WO 99/03861;
hypotensive active compounds, by way of example and with preference from the group of the calcium antagonists, angiotensin A11 antagonists, ACE inhibitors, renin inhibitors, beta-receptor blockers, alpha-receptor blockers, aldosterone antagonists, mineralocorticoid receptor antagonists, ECE inhibitors, ACE/NEP inhibitors and the vasopeptidase inhibitors; and/or
antithrombotic agents, by way of example and by way of preference from the group of the platelet aggregation inhibitors or the anticoagulants;
diuretics;
vasopressin receptor antagonists;
organic nitrates and NO donors;
compounds with positive inotropic activity;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil and tadalafil, and PDE 3 inhibitors such as milrinone;
natriuretic peptides, for example atrial natriuretic peptide (ANP, anaritide), B-type natriuretic peptide or brain natriuretic peptide (BNP, nesiritide), C-type natriuretic peptide (CNP) and urodilatin;
agonists of the prostacyclin receptor (IP receptor), such as, by way of example, iloprost, beraprost, cicaprost;
inhibitors of the If (funny channel) channel, such as, for example, ivabradine;
calcium sensitizers, a preferred example being levosimendan;
potassium supplements;
NO-independent but haem-dependent stimulators of guanylate cyclase, such as especially the compounds described in WO 00/06568, WO 00/06569, WO 02/42301 and WO 03/095451;
NO- and haem-independent activators of guanylate cyclase, such as especially the compounds described in WO 01/19355, WO 01/19776, WO 01/19778, WO 01/19780, WO 02/070462 and WO 02/070510;
inhibitors of human neutrophil elastase (HNE), for example sivelestat and DX-890 (Reltran);
compounds which inhibit the signal transduction cascade, for example tyrosine kinase inhibitors, especially sorafenib, imatinib, gefitinib and erlotinib; and/or
compounds which influence the energy metabolism of the heart, such as, for example, etomoxir, dichloroacetate, ranolazine and trimetazidine

Lipid metabolism-modifying active compounds are to be understood as meaning, preferably, compounds from the group of the HMG-CoA reductase inhibitors, squalene synthesis inhibitors, ACAT inhibitors, cholesterol absorption inhibitors, MTP inhibitors, lipase inhibitors, thyroid hormones and/or thyroid mimetics, niacin receptor agonists, CETP inhibitors, PPAR-α agonists, PPAR-γ agonists, PPAR-δ agonists, polymeric bile acid adsorbers, bile acid reabsorption inhibitors, antioxidants/radical scavengers and also the cannabinoid receptor 1 antagonists.

In a preferred embodiment of the invention, the compounds of the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494 or TAK-475.

In a preferred embodiment of the invention, the compounds of the invention are administered in combination with an ACAT inhibitor, by way of example and with preference avasimibe, melinamide, pactimibe, eflucimibe or SMP-797.

In a preferred embodiment of the invention, the compounds of the invention are administered in combination with a cholesterol absorption inhibitor, by way of example and with preference ezetimibe, tiqueside or pamaqueside.

In a preferred embodiment of the invention, the compounds of the invention are administered in combination with an MTP inhibitor, by way of example and with preference implitapide, BMS-201038, R-103757 or JTT-130.

In a preferred embodiment of the invention, the compounds of the invention are administered in combination with a lipase inhibitor, by way of example and with preference orlistat.

In a preferred embodiment of the invention, the compounds of the invention are administered in combination with a thyroid hormone and/or thyroid mimetic, by way of example and with preference D-thyroxine or 3,5,3′-triiodothyronine (T3).

In a preferred embodiment of the invention, the compounds of the invention are administered in combination with an agonist of the niacin receptor, by way of example and with preference niacin, acipimox, acifran or radecol.

In a preferred embodiment of the invention, the compounds of the invention are administered in combination with a CETP inhibitor, by way of example and with preference dalcetrapib, BAY 60-5521, anacetrapib or CETP vaccine (CETi-1).

In a preferred embodiment of the invention, the compounds of the invention are administered in combination with a PPAR-γ agonist from the class of the thiazolinediones, by way of example and with preference pioglitazone or rosiglitazone.

In a preferred embodiment of the invention, the compounds according to the invention are administered in combination with a PPAR-δ agonist, by way of example and with preference GW 501516 or BAY 68-5042.

In a preferred embodiment of the invention, the compounds of the invention are administered in combination with a polymeric bile acid adsorber, by way of example and with preference cholestyramine, colestipol, colesolvam, CholestaGel or colestimide.

In a preferred embodiment of the invention, the compounds of the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds of the invention are administered in combination with an antioxidant/radical scavenger, by way of example and with preference probucol, AGI-1067, BO-653 or AEOL-10150.

In a preferred embodiment of the invention, the compounds of the invention are administered in combination with a cannabinoid receptor 1 antagonist, by way of example and with preference rimonabant or SR-147778.

Antidiabetics are preferably understood as meaning insulin and insulin derivatives and also orally effective hypoglycemically active compounds. Here, insulin and insulin derivatives include both insulins of animal, human or biotechnological origin and mixtures thereof. The orally effective hypoglycaemically active compounds preferably include sulphonylureas, biguanides, meglitinide derivatives, glucosidase inhibitors and PPAR-gamma agonists.

In a preferred embodiment of the invention, the compounds of the invention are administered in combination with insulin.

In a preferred embodiment of the invention, the compounds of the invention are administered in combination with a sulphonylurea, by way of example and with preference tolbutamide, glibenclamide, glimepiride, glipizide or gliclazide.

In a preferred embodiment of the invention, the compounds of the invention are administered in combination with a biguanide, by way of example and with preference metformin.

In a preferred embodiment of the invention, the compounds of the invention are administered in combination with a meglitinide derivative, by way of example and with preference repaglinide or nateglinide.

In a preferred embodiment of the invention, the compounds of the invention are administered in combination with a glucosidase inhibitor, by way of example and with preference miglitol or acarbose.

In a preferred embodiment of the invention, the compounds of the invention are administered in combination with a DPP-IV inhibitor, by way of example and with preference sitagliptin and vildagliptin.

In a preferred embodiment of the invention, the compounds of the invention are administered in combination with a PPAR-gamma agonist from the class of the thiazolinediones, by way of example and with preference pioglitazone or rosiglitazone.

Hypotensive agents are preferably understood to mean compounds from the group of the calcium antagonists, angiotensin A11 antagonists, ACE inhibitors, beta-receptor blockers, alpha-receptor blockers and diuretics.

In a preferred embodiment of the invention, the compounds of the invention are administered in combination with a calcium antagonist, by way of example and with preference nifedipine, amlodipine, verapamil or diltiazem.

In a preferred embodiment of the invention, the compounds of the invention are administered in combination with an angiotensin A11 antagonist, by way of example and with preference losartan, valsartan, candesartan, embusartan, olmesartan or telmisartan.

In a preferred embodiment of the invention, the compounds of the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds of the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an alpha-receptor blocker, by way of example and with preference prazosin.

In a preferred embodiment of the invention, the compounds of the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

In a preferred embodiment of the invention, the compounds of the invention are administered in combination with an aldosterone or mineralocorticoid receptor antagonist, by way of example and with preference spironolactone or eplerenone.

In a preferred embodiment of the invention, the compounds of the invention are administered in combination with a vasopressin receptor antagonist, by way of example and with preference conivaptan, tolvaptan, lixivaptan or SR-121463.

In a preferred embodiment of the invention, the compounds of the invention are administered in combination with an organic nitrate or NO donor, by way of example and with preference sodium nitroprusside, nitroglycerol, isosorbide mononitrate, isosorbide dinitrate, molsidomine or SIN-1, or in combination with inhaled NO.

In a preferred embodiment of the invention, the compounds of the invention are administered in combination with a positive-inotropic compound, by way of example and with preference cardiac glycosides (digoxin), beta-adrenergic and dopaminergic agonists such as isoproterenol, adrenaline, noradrenaline, dopamine or dobutamine.

In a preferred embodiment of the invention, the compounds of the invention are administered in combination with antisympathotonics such as reserpine, clonidine or alpha-methyldopa, with potassium channel agonists such as minoxidil, diazoxide, dihydralazine or hydralazine, or with nitric oxide-releasing substances such as glycerol nitrate or sodium nitroprusside.

Antithrombotic agents are preferably understood to mean compounds from the group of the platelet aggregation inhibitors or the anticoagulants.

In a preferred embodiment of the invention, the compounds of the invention are administered in combination with a platelet aggregation inhibitor, by way of example and with preference aspirin, clopidogrel, ticlopidine or dipyridamole.

In a preferred embodiment of the invention, the compounds of the invention are administered in combination with a thrombin inhibitor, by way of example and with preference ximelagatran, melagatran, dabigatran, bivalirudin or clexane.

In a preferred embodiment of the invention, the compounds of the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or with a low molecular weight (LMW) heparin derivative.

In a preferred embodiment of the invention, the compounds of the invention are administered in combination with a vitamin K antagonist, by way of example and with preference coumarin.

In the context of the present invention, particular preference is given to combinations comprising at least one of the compounds according to the invention and one or more further active compounds selected from the group consisting of HMG-CoA reductase inhibitors (statins), diuretics, beta-receptor blockers, organic nitrates and NO donors, ACE inhibitors, angiotensin A11 antagonists, aldosterone and mineralocorticoid receptor antagonists, vasopressin receptor antagonists, platelet aggregation inhibitors and anticoagulants, and also their use for the treatment and/or prevention of the disorders mentioned above.

For the purpose of the present invention, the suitability for the treatment and/or prophylaxis of acute kidney disorders is to be understood as meaning in particular the suitability for the treatment and/or prophylaxis of acute renal insufficiency and of acute kidney failure (primary disorder and secondary disorder).

For the purpose of the present invention, the suitability for the treatment and/or prophylaxis of chronic kidney disorders is to be understood as meaning in particular the suitability for the treatment and/or prophylaxis of chronic renal insufficiency and of chronic kidney failure (primary disorder and secondary disorder).

For the purpose of the present invention, the term acute renal insufficiency encompasses acute manifestations of kidney disease, of kidney failure and/or renal insufficiency with and without the need for dialysis, and also underlying or related renal disorders such as renal hypoperfusion, intradialytic hypotension, volume deficiency (e.g. dehydration, blood loss), shock, acute glomerulonephritis, haemolytic-uraemic syndrome (HUS), vascular catastrophe (arterial or venous thrombosis or embolism), cholesterol embolism, acute Bence-Jones kidney in the event of plasmacytoma, acute supravesicular or subvesicular efflux obstructions, immunological renal disorders such as kidney transplant rejection, immune complex-induced renal disorders, tubular dilatation, hyperphosphataemia and/or acute renal disorders characterized by the need for dialysis, including in the case of partial resections of the kidney, dehydration through forced diuresis, uncontrolled blood pressure rise with malignant hypertension, urinary tract obstruction and infection and amyloidosis, and systemic disorders with glomerular factors, such as rheumatological-immunological systemic disorders, for example lupus erythematodes, renal artery thrombosis, renal vein thrombosis, analgesic nephropathy and renal tubular acidosis, and X-ray contrast agent- and medicament-induced acute interstitial renal disorders.

For the purpose of the present invention, the term chronic renal insufficiency encompasses chronic manifestations of kidney disease, of kidney failure and/or renal insufficiency with and without the need for dialysis, and also underlying or related renal disorders such as renal hypoperfusion, intradialytic hypotension, obstructive uropathy, glomerulopathy, glomerular and tubular proteinuria, renal edema, hematuria, primary, secondary and chronic glomerulonephritis, membranous and membranoproliferative glomerulonephritis, Alport syndrome, glomerulosclerosis, tubulointerstitial disorders, nephropathic disorders such as primary and congenital kidney disease, renal inflammation, immunological renal disorders such as kidney transplant rejection, immune complex-induced renal disorders, diabetic and non-diabetic nephropathy, pyelonephritis, renal cysts, nephrosclerosis, hypertensive nephrosclerosis and nephrotic syndrome, which can be characterized diagnostically, for example, by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, glomerular and arteriolar lesions, tubular dilatation, hyperphosphatemia and/or the need for dialysis, furthermore chronic kidney failure in the event of renal cell carcinoma, after partial resections of the kidney, dehydration through forced diuresis, uncontrolled blood pressure rise with malignant hypertension, urinary tract obstruction and infection and amyloidosis, and systemic disorders with glomerular factors, such as rheumatological-immunological systemic disorders, for example lupus erythematodes, and also renal artery stenosis, renal artery thrombosis, renal vein thrombosis, analgesic nephropathy and renal tubular acidosis. In addition chronic renal insufficiency owing to X-ray contrast agent- and medicament-induced chronic interstitial renal disorders, metabolic syndrome and dyslipidemia. The present invention also encompasses the use of the compounds of the invention for the treatment and/or prophylaxis of sequelae of renal insufficiency, for example pulmonary oedema, heart failure, uraemia, anaemia, electrolyte disorders (for example hyperkalaemia, hyponatraemia) and disorders in bone and carbohydrate metabolism.

The present invention further provides medicaments which comprise at least one compound of the invention, typically together with one or more inert, nontoxic, pharmaceutically suitable excipients, and for the use thereof for the aforementioned purposes.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which work according to the prior art and release the compounds of the invention rapidly and/or in a modified manner and which contain the compounds of the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Oral and parenteral administration are preferred, especially oral and intravenous administration.

The compounds of the invention can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration the dosage is about 0.01 to 100 mg/kg, preferably about 0.01 to 20 mg/kg and most preferably 0.1 to 10 mg/kg of body weight.

It may nevertheless be necessary in some cases to deviate from the stated amounts, specifically as a function of body weight, route of administration, individual response to the active ingredient, nature of the preparation and time or interval over which administration takes place. Thus, in some cases less than the abovementioned minimum amount may be sufficient, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight. Solvent ratios, dilution ratios and concentration data for the liquid/liquid solutions are based in each case on volume.

### EXPERIMENTAL PART

Abbreviations and Acronyms

DCC dicyclohexylcarbodiimide

DMAP 4-(dimethylamino)pyridine

g gram

h hour

HPLC high pressure liquid chromatography

kg kilogram

l litre

min minute

MS mass from mass spectrometry

THF tetrahydrofuran

### EXAMPLES

### Example 1

### 2-Amino-6-({[2-(4-chlorophenyl)-1,3-thiazol-4-yl]methyl}sulphanyl)-4-[4-(2-hydroxyethoxy)phenyl]pyridine-3,5-dicarbonitrile (II)

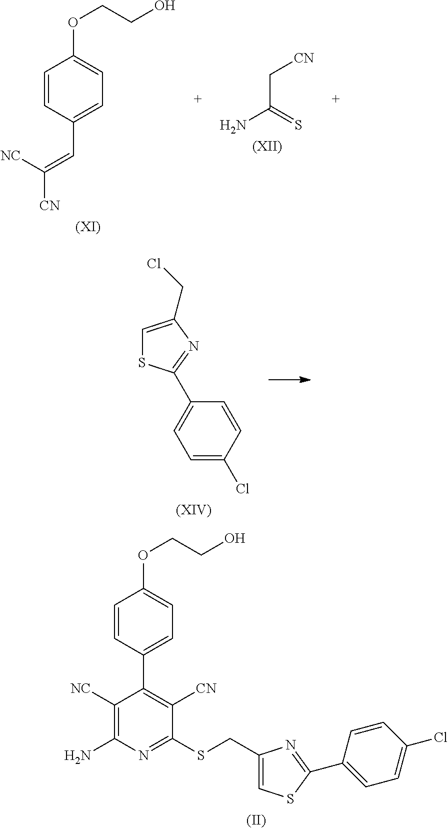

5.334 kg (24.90 mol) of 2-[4-(2-hydroxyethoxy)benzylidene]malononitrile (XI) and 1.309 kg (13.07 mol) of 2-cyanothioacetamide (XII) were suspended in 27.4 kg (34.8 l) of methanol. The suspension was warmed to 40° C., and 3.779 kg (37.35 mol) of triethylamine were metered in at at most 40° C. The mixture was stirred at 40° C. for another 3 h and cooled to room temperature. 3.147 kg (12.45 mol) of 4-(chloromethyl)-2-(4-chlorophenyl)-1,3-thiazole (XIV) were added to the dark-brown solution, and the content of the tank was stirred at room temperature overnight. The suspension now present was cooled to 5° C., isolated by filtration and washed with 11.7 kg (14.85 l) of methanol in total. The moist product was dried at 50° C. in a vacuum drying cabinet.

This gave 4862 g or 75.1% of theory of 2-amino-6-({[2-(4-chlorophenyl)-1,3-thiazol-4-yl]methyl}sulphanyl)-4-[4-(2-hydroxyethoxy)phenyl]pyridine-3,5-dicarbonitrile as a beige-greenish solid (content 95.5%, ESTD).

HPLC method A: Retention time about 14.1 min.

### Example 2

### 2-Chloro-6-({[2-(4-chlorophenyl)-1,3-thiazol-4-yl]methyl}sulphanyl)-4-[4-(2-hydroxyethoxy)phenyl]pyridine-3,5-dicarbonitrile (III)

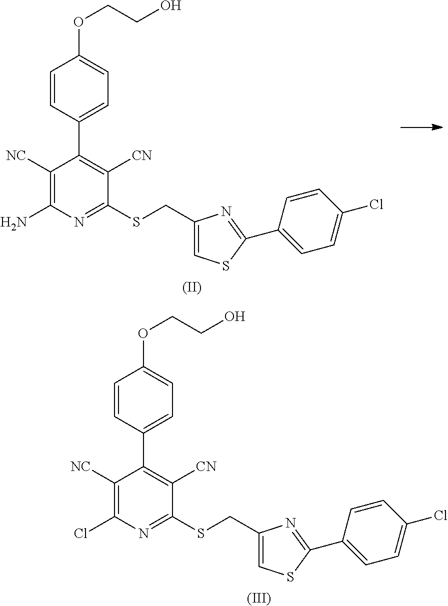

At room temperature, 1.75 kg (7.68 mol) of benzyltriethylammonium chloride were dissolved in 14.0 kg (17.8 l) of acetonitrile. 2.0 kg (3.846 mol) of 2-amino-6-({[2-(4-chlorophenyl)-1,3-thiazol-4-yl]methyl}sulphanyl)-4-[4-(2-hydroxyethoxy)phenyl]pyridine-3,5-dicarbonitrile (II) were added. 1.66 kg (12.35 mol) of copper(II) chloride were then added to the suspension, rinsing with 1.00 kg (1.27 l) of acetonitrile, and the mixture was warmed to 50° C. At this temperature, 793 g (7.69 mol) of tert-butyl nitrite were metered in, rinsing with 1.00 kg (1.27 l) of acetonitrile. The content of the tank was stirred at 50° C. for another 3 h, cooled to room temperature and stirred with 9.0 kg (10.0 l) of ethyl acetate and 10 kg (10 l) of water. After settling, the aqueous phase was separated off and the organic phase was once more stirred with 10 kg (10 l) of water. After settling again, the aqueous phase was removed. A third time, the organic phase was washed with 10 kg (10 l) of water, with addition of a further 3.6 kg (4.0 l) of ethyl acetate. At 300 mbar and an internal temperature of at most 42° C., 15.5 l of distillate were removed from the organic phase. 9.6 kg (12.1 l) of methanol were added to the suspension that remained, and the content of the tank was stirred at reflux (about 67° C.) for 1 h. The resulting suspension was filtered with suction and washed with 8 kg (10.1 l) of methanol in total. The moist product was dried overnight at 50° C. in a vacuum drying cabinet until the weight remained constant.

This gave 1636 g or 78.8% of theory of 2-chloro-6-({[2-(4-chlorophenyl)-1,3-thiazol-4-yl]methyl}sulphanyl)-4-[4-(2-hydroxyethoxy)phenyl]pyridine-3,5-dicarbonitrile as an ochre powder.

### Example 3

### 2-({[2-(4-Chlorophenyl)-1,3-thiazol-4-yl]methyl}sulphanyl)-4-[4-(2-hydroxyethoxy)phenyl]-6-(pyrrolidin-1-yl)pyridine- 3,5-dicarbonitrile (IV)

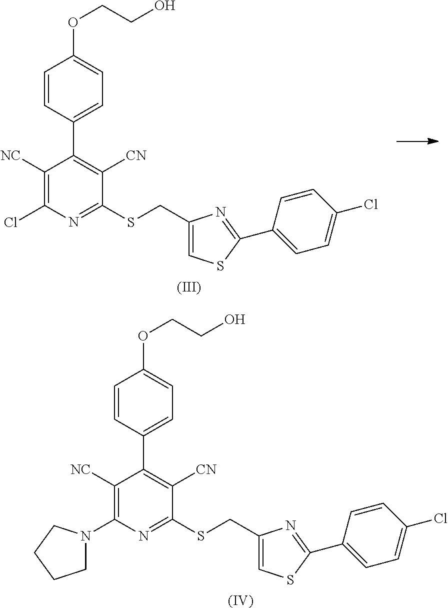

1.60 kg (2.966 mol) of 2-chloro-6-({[2-(4-chlorophenyl)-1,3-thiazol-4-yl]methyl}sulphanyl)-4-[4-(2- hydroxyethoxy)phenyl]pyridine-3,5-dicarbonitrile (III) were suspended in 27.2 kg (34.4 l) of methanol. Over a period of 30 min, 1.58 kg (22.24 mol) of pyrrolidine were metered in such that the internal temperature did not exceed 30° C. The mixture was subsequently stirred at room temperature overnight and then at reflux (65-66° C.) for 1 h. The mixture was cooled to room temperature and the solid was filtered off and washed with 7.6 kg (9.6 l) of methanol in total. The moist product was dried at 50° C. under reduced pressure.

This gave 1549 g or 91.0% of theory of 2-({[2-(4-chlorophenyl)-1,3-thiazol-4-yl]methyl}sulphanyl)-4-[4-(2- hydroxyethoxy)phenyl]-6-(pyrrolidin-1-yl)pyridine-3,5-dicarbonitrile as a beige powder.

HPLC method B: Retention time about 12.0 min.

Typically, the following byproducts are obtained:

N-{6-({[2-(4-Chlorophenyl)-1,3-thiazol-4-yl]methyl}sulphanyl)-3,5-dicyano-4-[4-(hydroxyethoxy)phenyl]-pyridin-2-yl}acetamide (“aminoacetylmercaptothiazolopyridine”) having a typical content of 0.2%. HPLC method B: relative retention time 0.56 (about 7.3 min).

2-Amino-6-({[2-(4-chlorophenyl)-1,3-thiazol-4-yl]methyl}sulphanyl)-4-[4-(2-hydroxyethoxy)phenyl]pyridine-3,5-dicarbonitrile (II) having a typical content of 0.3%.

HPLC method B: relative retention time 0.62 (about 8.1 min).

2-({[2-(4-Chlorophenyl)-1,3-thiazol-4-yl]methyl}sulphanyl)-6-hydroxy-4-[4-(2-hydroxyethoxy)phenyl]pyridine-3,5-dicarbonitrile (“hydroxymercapthiazolopyridine”) having a typical content of 0.3%.

HPLC method B: relative retention time 1.32 (about 17.3 min).

### Example 4

### 2-({[2-(4-Chlorophenyl)-1,3-thiazol-4-yl]methyl}sulphanyl)-4-[4-(2-hydroxyethoxy)phenyl]-6-(pyrrolidin-1-yl)pyridine- 3,5-dicarbonitrile (IV)

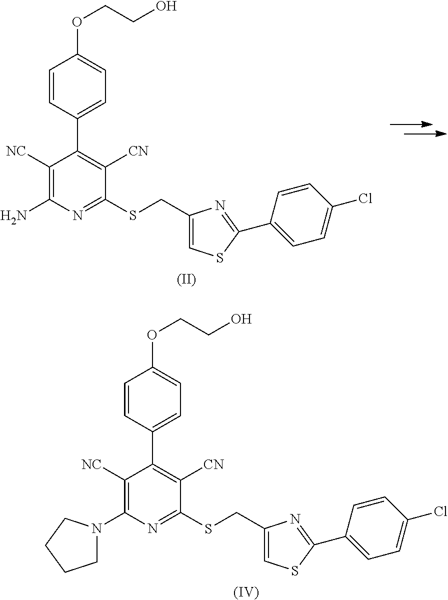

1. Partial Step

At room temperature, 1.23 kg (5.38 mol) of benzyltriethylammonium chloride were dissolved in 10.9 kg (13.9 l) of acetonitrile. 1.40 kg (2.69 mol) of 2-amino-6-({[2-(4-chlorophenyl)-1,3-thiazol-4-yl]methyl}sulphanyl)-4-[4-(2-hydroxyethoxy)phenyl]pyridine-3,5-dicarbonitrile (II) were added. 1.16 kg (8.62 mol) of copper(II) chloride were then added to the suspension, rinsing with 0.42 kg (0.523 l) of acetonitrile, and the mixture was warmed to 50° C. At this temperature, 0.56 kg (5.38 mol) of tert-butyl nitrite (about 90% pure, purity not taken into account in calculations) were metered in, rinsing with 0.28 kg (0.36 l) of acetonitrile. The content of the tank was stirred at 50° C. for another 4 h. The mixture was cooled to room temperature and stirred with 17.0 kg (19.6 l) of isopropyl acetate and 7.25 kg (13.9 mol) of 7% strength hydrochloric acid. After settling, the aqueous phase was separated off and the organic phase was once more stirred with 7.25 kg (13.9 mol) of 7% strength hydrochloric acid. After settling again (10 min), the aqueous phase was removed. A third time, the organic phase was stirred with 8.4 kg (7.0 l) of saturated sodium chloride solution. The phases were then separated, and the lower aqueous phase was removed. The light-brown organic upper phase was filtered through a filter plate to remove precipitated fines. Here, 0.54 kg (0.62 l) of isopropyl acetate were used for rinsing and washing. The solution obtained in this manner was used in the next step without further treatment.

27.34 kg of solution were weighted out. Based on the 2.69 mol of starting material employed, the solution theoretically contained 1.45 kg of 2-chloro-6-({[2-(4-chlorophenyl)-1,3-thiazol-4-yl]methyl}sulphanyl)-4-[4-(2-hydroxyethoxy)phenyl]pyridine-3,5-dicarbonitrile.

2. Partial Step

5.51 kg (7.01 l) of methanol were added to the solution, prepared in the first partial step, of 27.34 kg (2.69 mol) of 2-chloro-6-({[2-(4-chlorophenyl)-1,3-thiazol-4-yl]methyl}sulphanyl)-4-[4-(2-hydroxyethoxy)phenyl]pyridine-3,5-dicarbonitrile in isopropyl acetate, and 0.77 kg (10.77 mol) of pyrrolidine were metered in. By cooling, the addition was carried out at at most 25° C. The mixture was stirred at room temperature for another 2 h and heated at reflux (65-66° C.) overnight. The mixture was cooled to about 20° C., stirred at about 20° C. for 2 h, cooled further to about 5° C. and stirred for another 1 h. The product was then filtered off with suction, rinsed and washed with 5.34 kg (6.8 l) of methanol in total. The moist product (1.42 kg) was dried at 50° C. under reduced pressure.

This gave 1300 g (2.26 mol) or 84.1% of theory over the two steps of 2-({[2-(4-chlorophenyl)-1,3-thiazol-4-yl]methyl}sulphanyl)-4-[4-(2-hydroxyethoxy)phenyl]-6-(pyrrolidin-1-yl)pyridine-3,5-dicarbonitrile as a beige-yellow powder.

### Example 5

### 2-{4-[2-({[2-(4-Chlorophenyl)-1,3-thiazol-4-yl]methyl}sulfanyl)-3,5-dicyano-6-(pyrrolidin-1-yl)pyridin-4-yl]phenoxy}ethyl L-alaninate dihydrochloride (VIII)

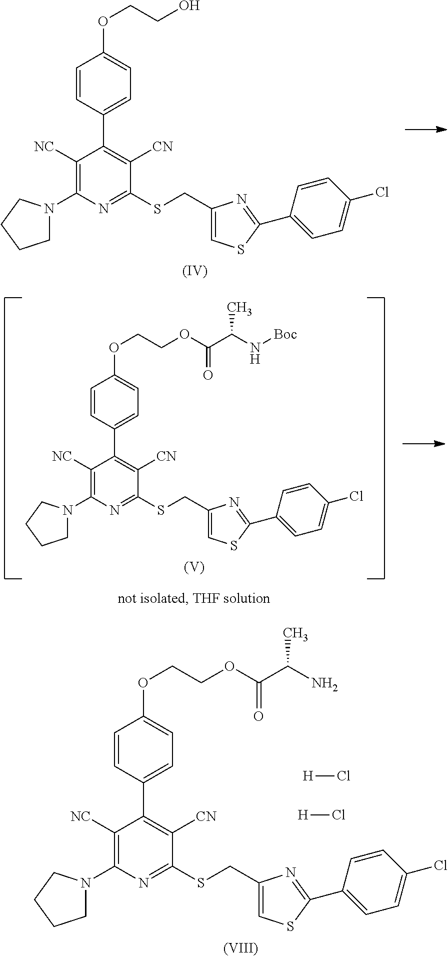

1. Partial Step

1.250 kg (2.177 mol) of 2-({[2-(4-chlorophenyl)-1,3-thiazol-4-yl]methyl}sulphanyl)-4-[4-(2-hydroxyethoxy)-phenyl]-6-(pyrrolidin-1-yl)pyridine-3,5-dicarbonitrile (IV), 577 g (3.048 mol) of Boc-L-alanine and 8.25 kg (9.4 l) of THF were initially charged. 133 g (1.093 mol) of DMAP were added to the suspension, and a solution of 674 g (3.268 mol) of DCC and 2.52 kg (2.9 l) of THF was then metered in at about 20° C. The content of the tank was stirred at room temperature overnight. The precipitated dicyclohexylurea was filtered off with suction and washed with a total of 2.14 kg (2.4 l) of THF. Under reduced pressure, the resulting filtrate of about 14.5 kg was concentrated to an amount of 3.2 kg. Without further treatment, this solution of the Boc-L-alaninate (V) is employed for the next step.

2. Partial Step

3.2 kg (2.177 mol) of Boc-L-alaninate (V) solution from the first partial step were diluted with 11.9 kg of THF. 6.90 kg (26.15 mol) of a solution of 4 M HCl in dioxane were then metered in at an internal temperature of about 23° C. (20-25° C.). The content of the tank was finally stirred at about 23° C. (20-25° C.) for 12 h. The precipitated solid was filtered off with suction and washed with a total of 19.0 kg (21.0 l) of THF. The moist product was dried at 40° C. under reduced pressure for 20 h.

Over the two steps, 1719 g of 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl L-alaninate dihydrochloride were weighted out. Calculated for the molecular weight of 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl L-alaninate dihydrochloride, this gave a formal yield of 109% of theory. The formally calculated yield of more than 100% is due to reagent and solvent residues and essentially to dimethylaminopyridine hydrochloride, which is present in the isolated product. These impurities do not reduce the quality of the alaninate dihydrochloride intermediate (VIII), since all reagents are used again in the next step and the subsequent stages Boc-dialaninate (VII) and dialaninate dihydrochloride (IX) are purified completely from these impurities.

HPLC method C: Retention time about 13.1 min.

Typically, the following byproducts are obtained:

N,N-Dimethylpyridin-4-amine (dimethylaminopyridine) having a typical content of 8 to 10%.

HPLC method C: relative retention time 0.15 (about 1.8 min).

2-{4-[2-({[2-(4-Chlorophenyl)-1,3-thiazol-4-yl]methyl}sulphanyl)-3,5-dicyano-6-hydroxypyridin-4-yl]phenoxy}ethyl L-alaninate (“hydroxyalaninate”) having a typical content of 0.3%.

HPLC method C: relative retention time 0.77 (about 9.2 min).

2-({[2-(4-Chlorophenyl)-1,3-thiazol-4-yl]methyl}sulphanyl)-4-[4-(2-hydroxyethoxy)phenyl]-6-(pyrrolidin-1-yl)pyridine-3,5-dicarbonitrile (IV) having a typical content of 0.5 to 1%.

HPLC method C: relative retention time 1.71 (about 20.5 min).

### Example 6

### 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl N-(tert-butoxycarbonyl)-L-alanyl-L-alaninate (VII)

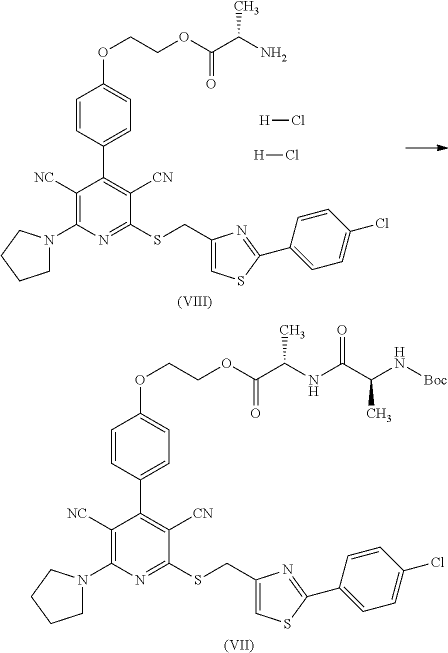

1.650 kg (2.298 mol) of 2-{4-[2-({[2-(4-chlorophenyl)-1,3-thiazol-4-yl]methyl}sulphanyl)-3,5-dicyano-6- (pyrrolidin-1-yl)pyridin-4-yl]phenoxy}ethyl L-alaninate dihydrochloride (VIII), 14.7 kg of THF and 562 g (4.599 mol) of DMAP were initially charged and stirred at 20-25° C. 522 g (2.757 mol) of Boc-L-alanine were then added at 20-25° C. A solution of 570 g (2.761 mol) of DCC and 3.10 kg of THF was then metered in, rinsing with 200 g of THF, and the mixture was stirred at 20-25° C. for 4 h. The precipitated urea was filtered off, rinsing with 2.60 kg of THF in total. The resulting solution was washed three times with in each case 5.2 kg of an aqueous 15% strength solution of ammonium chloride in water. At 200 mbar and an internal temperature of at most 50° C., an amount of 9.4 kg of distillate was removed from the water-moist organic phase. 7.7 kg of dioxane were added to the bottom that remained, and another amount of 10.2 kg of distillate was removed at 200 mbar at an internal temperature of at most 60° C. Another 7.7 kg of dioxane were added to the bottom, and an amount of 7.9 kg of distillate was removed at 200 mbar at an internal temperature of at most 70° C. A third time, 7.7 kg of dioxane were added to the bottom, and a final amount of 7.9 kg of distillate was removed at 200 mbar at an internal temperature of at most 70° C. Finally, 6.7 kg of dioxane were added to the bottom, and the content of the flask was filtered to remove small amounts of solids.

At 20-25° C. and with stirring, the filtrate was metered into an amount of 22.3 kg of diisopropyl ether which had initially been charged. The filtrate was rinsed in with 1.1 kg of dioxane. The precipitated solid was filtered off at 20-25° C. The solid was washed with 8.4 kg of diisopropyl ether. At room temperature, the moist cake was stirred for about 1 h with 5.2 kg of ethanol and then filtered off and washed with 4.9 kg of ethanol. The moist product was dried in a drying cabinet at a jacket temperature of 50° C. under reduced pressure.

This gave 1403 g. Without taking the content of the alaninate dihydrochloride (VIII) into account, this corresponded to a yield of 74.8% of theory of 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-N-(tert-butoxycarbonyl)-L-alanyl L-alaninate.

HPLC method D: Retention time about 11.9 min.

Typically, the following byproducts are obtained:

2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl N-(tert-butoxycarbonyl)-L-alanyl-L-alanyl-L-alaninate (“Boc-trialaninate”) having a typical content of 0.4%.

HPLC method D: relative retention time 0.89 (about 10.6 min).

2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl N-(tert-butoxycarbonyl)-L-alaninate (“Boc-alaninate”, V) having a typical content of 0.3%.

HPLC method D: relative retention time 1.19 (about 14.1 min).

### Example 7

2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl L-alanyl-L-alaninate dihydrochloride (IX)

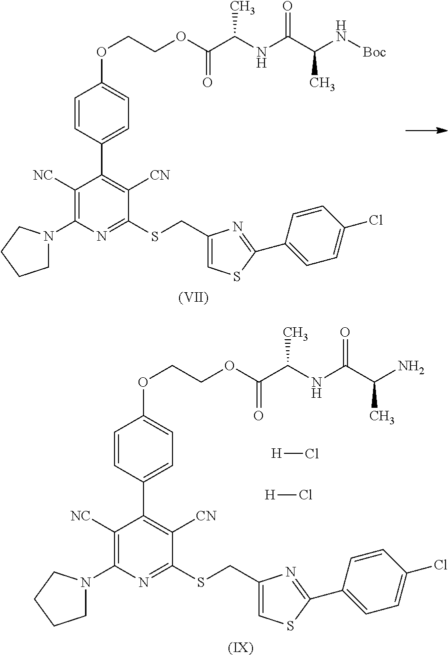

1440 g (1.76 mol) of 2-{4-[2-({[2-(4-chlorophenyl)-1,3-thiazol-4-yl]methyl}sulphanyl)-3,5-dicyano-6-(pyrrolidin- 1-yl)pyridin-4-yl]phenoxy}ethyl N-(tert-butoxycarbonyl)-L-alanyl-L-alaninate (VII) were initially charged. 26.0 kg of dichloromethane were added and the mixture was, with stirring, warmed to 40° C. The slightly turbid solution was filtered whilst still warm, rinsing with 2.0 kg of dichloromethane. At atmospheric pressure, about 16.6 kg (about 12.5 l) of distillate were then removed at a prerun temperature of at most 60° C. The bottom that remained was diluted with 3.5 kg of diisopropyl ether. 5.56 kg (21.2 mol) of 4 M HCl in dioxane were metered in at 20-25° C., rinsing with 200 g of dioxane, and the mixture was stirred at room temperature overnight (16 h). The precipitated colourless solid was filtered off with suction and washed with 18.6 kg of dichloromethane in three portions of equal size. The cake was then washed with 13.5 kg of isopropanol. The moist cake was then removed and dried in a vacuum drying cabinet at 50° C. for 6 h.

This gave 1349 g or 96.9% of theory of 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl L-alanyl-L-alaninate dihydrochloride.

### Example 8

### 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl L-alanyl-L-alaninate monohydrochloride (I)

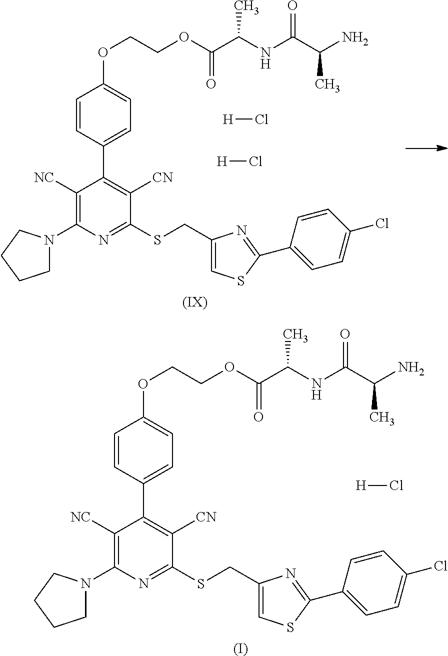

The total amount weighted out of 1349 g of dried 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl L-alanyl-L-alaninate dihydrochloride (IX) was stirred in 21 kg of isopropanol/water (98:2 v/v) at room temperature for 67 h and then filtered and washed with 9.6 kg of isopropanol. In a vacuum drying cabinet, the product was dried for 16 h at 50° C. until the weight remained constant.

This gave 1185 g (1.57 mol) or 88.2% of theory [based on the amount of protected Boc-dialaninate (VII) employed] or 91.8% of theory [based on the dialaninate dihydrochloride (IX)] of 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl L-alanyl-L-alaninate monohydrochloride as a colourless powder.

HPLC method E: Retention time about 15.1 min.

Typically, the following byproducts are obtained:

2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl L-alanyl-L-alanyl-L-alaninate (“trialaninate”) having a typical content of 0.4%. HPLC method E: relative retention time 0.88 (about 13.3 min).

rac 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl L-alanyl-D-alaninate (“D,L-dialaninate”) having a typical content of 0.1%.

HPLC method E: relative retention time 1.07 (about 16.2 min).

2-{4-[2({[2-(4-Chlorophenyl)-1,3-thiazol-4-yl]methyl}sulphanyl)-3,5-dicyano-6-(pyrrolidin-1-yl)pyridin-4-yl]phenoxy}ethyl L-alaninate (“alaninate”) having a typical content of 0.3%.

HPLC method E: relative retention time 1.35 (about 20.4 min).

2-({[2-(4-Chlorophenyl)-1,3-thiazol-4-yl]methyl}sulphanyl)-4-[4-(2-hydroxyethoxy)phenyl]-6-(pyrrolidin-1-yl)pyridine-3,5-dicarbonitrile (V) having a typical content of 0.3%.

HPLC method E: relative retention time 1.60 (about 24.1 min).

2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl N-(tert-butoxycarbonyl)-L-alanyl-L-alaninate (“Boc-dialaninate”) having a typical content of 0.1%.

HPLC method E: relative retention time 2.35 (about 35.5 min).

### Example 9

### 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate dihydrochloride (IX)

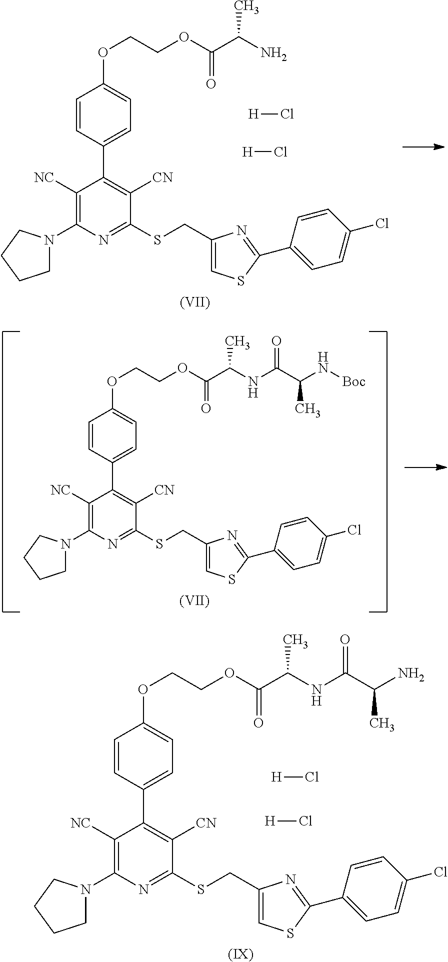

1. Partial Step

1.37 kg (1.908 mol) of 2-{4-[2-({[2-(4-chlorophenyl)-1,3-thiazol-4-yl]methyl}sulphanyl)-3,5-dicyano-6- (pyrrolidin-1-yl)pyridin-4-yl]phenoxy}ethyl L-alaninate dihydrochloride (VIII), 12.2 kg of THF and 466 g (3.813 mol) of DMAP were initially charged and stirred at 20-25° C. 432 g (2.281 mol) of Boc-L-alanine were then added at 20-25° C. A solution of 473 g (2.291 mol) of DCC and 2.33 kg of THF was then metered in, rinsing with 400 g of THF, and the mixture was stirred at 20-25° C. for 4 h. The precipitated urea was filtered off, rinsing with 2.40 kg of THF in total. The resulting solution was washed three times with in each case 4.3 kg of an aqueous 15% strength solution of ammonium chloride in water. At 200 mbar and an internal temperature of at most 50° C., an amount of 8.2 kg of distillate was removed from the organic phase. 6.9 kg of dioxane were added to the bottom that remained, and another amount of 9.6 kg of distillate was removed at 200 mbar at an internal temperature of at most 60° C. Another 6.9 kg of dioxane were added to the bottom, and an amount of 6.9 kg of distillate was removed at 200 mbar at an internal temperature of at most 70° C. A third time, 6.9 kg of dioxane were added to the bottom, and a final amount of 6.2 kg of distillate was removed at 200 mbar at an internal temperature of at most 70° C. Finally, 19.0 kg of dioxane were added to the bottom, and the content of the flask was filtered to remove small amounts of solids, rinsing with 3.6 kg of dioxane.

The resulting solution (about 24.2 kg) was used in this form for the 2. partial step.

2. Partial Step

24.2 kg (1.908 mol) of the solution from the 1. partial step were initially charged and stirred at 20° C. 6.01 kg (22.89 mol) of an about 4.0 M solution of HCl in dioxane were metered in at an internal temperature of about 20° C., and the mixture was stirred at room temperature overnight. The content of the flask was then filtered, rinsing and washing with 10.1 kg of dioxane in total, and dried at 50° C. under reduced pressure.

### Example 10

### 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl L-alanyl-L-alaninate hydrochloride—emulation of Example 44 from WO 2010/086101

1.5 g (1.837 mmol) of the protected Boc-dialaninate (VII) were initially charged in 24 ml of dichloromethane. The mixture was briefly heated to reflux, allowed to cool to room temperature and, after addition of 18.37 ml of a IN solution of hydrogen chloride in diethyl ether, stirred at room temperature overnight. The resulting solid was filtered off with suction and washed twice with in each case about 20 ml of diethyl ether. Drying under reduced pressure at 50° C. overnight gave 1.43 g of the target compound.

The chloride determination showed a content of 7.8 percent by weight, which corresponds to about 1.7 mol of HCl per mol of the heterocyclic parent substance.

The X-ray diffractogram of this target compound was obtained under the conditions mentioned below (see FIG. 5).

HPLC Conditions/Methods

Method A

Zorbax Bonus RP; 150×3 mm; 3.5 μm

Oven temperature: 40° C.; injection volume: 2 μl; flow rate: 0.5 ml/min

mobile phase A: 1.36 g of potassium dihydrogenphosphate+1.15 g of conc. phosphoric acid (85% in water)/1 l of water;

mobile phase B: acetonitrile;

linear stepped gradient: 0′: 10% B; 12′: 66% B; 25′: 80% B; 35′: 80% B

UV detection: 0′ to 4′: 265 nm, 4′ to 35′: 300 nm.

Method B

Zorbax Bonus RP; 150×3 mm; 3.5 μm

Oven temperature: 40° C.; injection volume: 2 μl; flow rate: 0.7 ml/min

mobile phase A: 1.36 g of potassium dihydrogenphosphate+1.15 g of conc. phosphoric acid (85% in water)/1 l of water;

mobile phase B: acetonitrile;

linear stepped gradient: 0′: 50% B; 20′: 80% B; 50′: 80% B

UV detection: 300 nm.

Method C

Zorbax Bonus RP; 150×3 mm; 3.5 μm

Oven temperature: 40° C.; injection volume: 3 μl; flow rate: 0.7 ml/min

mobile phase A: 1.36 g of potassium dihydrogenphosphate+1.15 g of conc. phosphoric acid (85% in water)+1 g of hexanesulphonic acid sodium salt/1 l of water;

mobile phase B: 50 ml of methanol/1 l of acetonitrile;

linear stepped gradient: 0′: 10% B; 5′: 40% B; 13′: 50% B; 20′: 75% B; 30′: 80% B; 35′: 80% B

UV detection: 250 nm.

Method D

Zorbax RRHD Eclipse Plus C8; 100×2.1 mm; 1.8 μm

Oven temperature: 30° C.; injection volume: 2 μl; flow rate: 0.5 ml/min

Mobile phase A: 0.5 ml of conc. phosphoric acid (85% in water)/1 l of water;

mobile phase B: 300 ml of methanol/1 l of acetonitrile;

linear stepped gradient: 0′: 60% B; 20′: 80% B; 30′: 95% B

UV detection: 290 nm.

Method E

Zorbax YMC Triart C18; 100×3 mm; 1.9 μm

Oven temperature: 20° C.; injection volume: 3 μl; flow rate: 0.6 ml/min

mobile phase A: 1.5 g of ammonium acetate+3.5 ml of 1% strength acetic acid in water/1 l of water;

mobile phase B: 50 ml of methanol/1 l of acetonitrile;

linear stepped gradient: 0′: 53% B; 30′: 70% B; 40′: 75% B; 45′: 75% B

UV detection: 300 nm.

Measuring Parameters of the X-Ray Diffractometry for the Measurement of Compound of the Formula (I) in Crystalline Form of Modifications I, II, III and IV

| | |
|---|---|
| Scan axis | 2 theta |
| Measurement temperature [° C.] | 25 |
| Anode material | Cu |
| K-alpha1 [Å] | 1.54060 |
| Generator setting | 40 mA, 40 kV |
| Diffractometer type | transmission diffractometer |
| Primary beam monochromator | yes |
| Sample rotation | yes |

TABLE 1
Peak maxima of the 2 theta angle
Peak maximum [2 theta]

| Modification I | Modification II | Modification III | Modification IV |
|---|---|---|---|
| 4.3 | 3.7 | 3.1 | 3.0 |
| 5.4 | 4.0 | 6.2 | 6.0 |
| 6.5 | 6.4 | 8.0 | 9.0 |
| 8.7 | 7.5 | 10.0 | 12.1 |
| 9.8 | 9.1 | 10.4 | 13.7 |
| 10.8 | 9.5 | 12.3 | 15.3 |
| 11.8 | 10.5 | 13.2 | 17.0 |
| 13.0 | 12.5 | 15.2 | 20.8 |
| 13.9 | 13.7 | 15.7 | 21.3 |
| 14.9 | 14.1 | 16.5 | 22.8 |
| 15.4 | 14.8 | 18.3 | 25.2 |
| 16.0 | 15.1 | 19.2 | 27.5 |
| 16.3 | 15.7 | 19.8 | 29.2 |
| 17.1 | 16.3 | 21.1 | 31.0 |
| 18.2 | 17.0 | 22.0 | |
| 18.3 | 17.5 | 22.7 | |
| 18.9 | 19.3 | 24.0 | |
| 19.7 | 19.7 | 26.0 | |
| 19.9 | 22.8 | 27.0 | |
| 20.2 | 23.8 | 29.7 | |
| 20.7 | 24.9 | 31.5 | |
| 21.5 | 25.4 | 32.5 | |
| 22.6 | 26.7 | 33.7 | |
| 23.0 | 27.5 | | |
| 23.5 | 34.7 | | |
| 23.7 | | | |
| 24.3 | | | |
| 24.9 | | | |
| 25.6 | | | |
| 26.3 | | | |
| 27.4 | | | |
| 28.4 | | | |
| 29.3 | | | |
| 30.3 | | | |
| 31.6 | | | |
| 32.9 | | | |
| 35.8 | | | |
| 37.3 | | | |

Raman Spectroscopy Measurement Conditions for Measuring the Compound of the Formula (I) in the Crystalline Form of Modifications I, II, III and IV and in the Form of the Isopropanol Solvate:

| Instrument | Bruker Raman RFS 100/S | |
|---|---|---|
| Number of scans | 64 | |
| Resolution | 2-4 | cm⁻¹ |
| Laser power | 50 | mW |
| Laser wavelength | 1064 | nm |

TABLE 2
Band maxima [cm⁻¹] in the Raman spectrum
Band maxima [cm⁻¹]

| Modification I | Modification II | Modification III | Modification IV | Isopropanol solvate |
|---|---|---|---|---|
| 3102 | 3066 | 3071 | 3073 | 3090 |
| 3069 | 2944 | 2973 | 2988 | 3074 |
| 2965 | 2212 | 2946 | 2942 | 2987 |
| 2939 | 1607 | 2879 | 2883 | 2939 |
| 2907 | 1595 | 2217 | 2212 | 2880 |
| 2211 | 1534 | 1609 | 1610 | 2209 |
| 1610 | 1501 | 1596 | 1597 | 1728 |
| 1596 | 1454 | 1546 | 1523 | 1666 |
| 1539 | 1401 | 1525 | 1503 | 1609 |
| 1515 | 1323 | 1497 | 1451 | 1595 |
| 1498 | 1241 | 1449 | 1400 | 1551 |
| 1447 | 1188 | 1400 | 1326 | 1539 |
| 1402 | 1135 | 1326 | 1224 | 1526 |
| 1394 | 1093 | 1294 | 1186 | 1517 |
| 1338 | 739 | 1267 | 1112 | 1495 |
| 1325 | 650 | 1185 | 1093 | 1447 |
| 1295 | 526 | 1162 | 737 | 1399 |
| 1245 | 419 | 1133 | 650 | 1391 |
| 1225 | | 1090 | 629 | 1339 |
| 1182 | | 774 | 529 | 1320 |
| 1163 | | 737 | 444 | 1295 |
| 1133 | | 647 | 417 | 1253 |
| 1095 | | 627 | 361 | 1231 |
| 1018 | | 531 | 322 | 1174 |
| 1004 | | 506 | | 1136 |
| 883 | | 424 | | 1092 |
| 783 | | 362 | | 1015 |
| 736 | | 317 | | 956 |
| 646 | | 272 | | 939 |
| 627 | | | | 921 |
| 598 | | | | 879 |
| 527 | | | | 819 |
| 506 | | | | 781 |
| 420 | | | | 763 |
| 361 | | | | 734 |
| 337 | | | | 645 |
| 315 | | | | 627 |
| 294 | | | | 529 |
| 270 | | | | 505 |
| 227 | | | | 453 |
| | | | | 421 |
| | | | | 361 |
| | | | | 328 |
| | | | | 315 |
| | | | | 267 |
| | | | | 225 |
| | | | | 120 |
| | | | | 163 |

### DESCRIPTION OF THE FIGURES

FIG. 1: X-ray diffractogram of the compound of the formula (I) in crystalline form of modification I

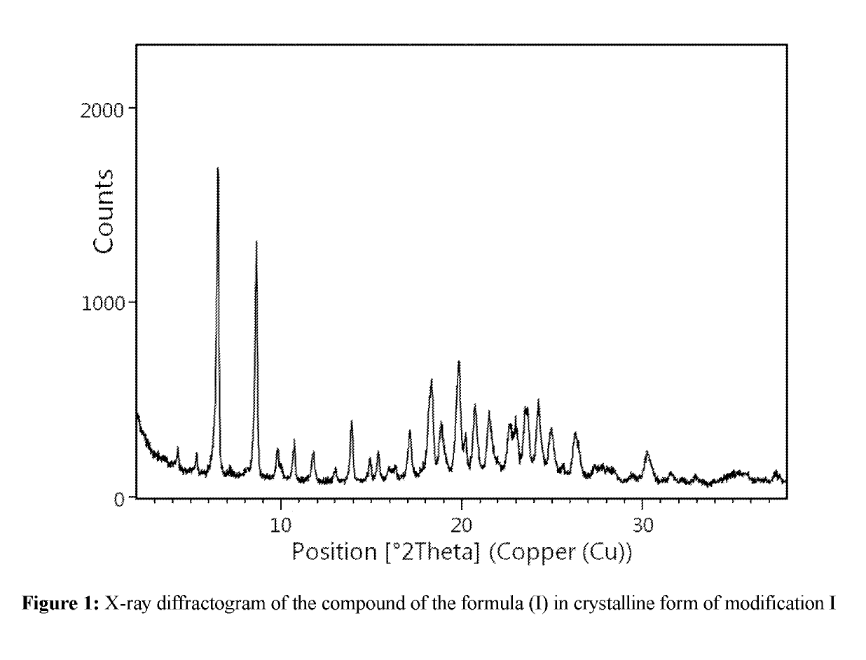

FIG. 2: X-ray diffractogram of the compound of the formula (I) in crystalline form of modification II

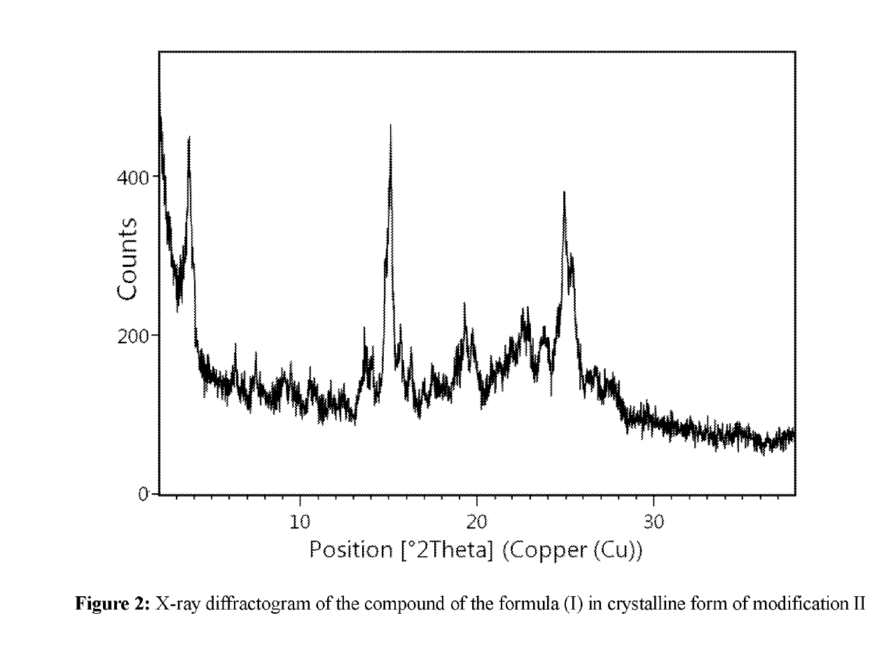

FIG. 3: X-ray diffractogram of the compound of the formula (I) in crystalline form of modification III

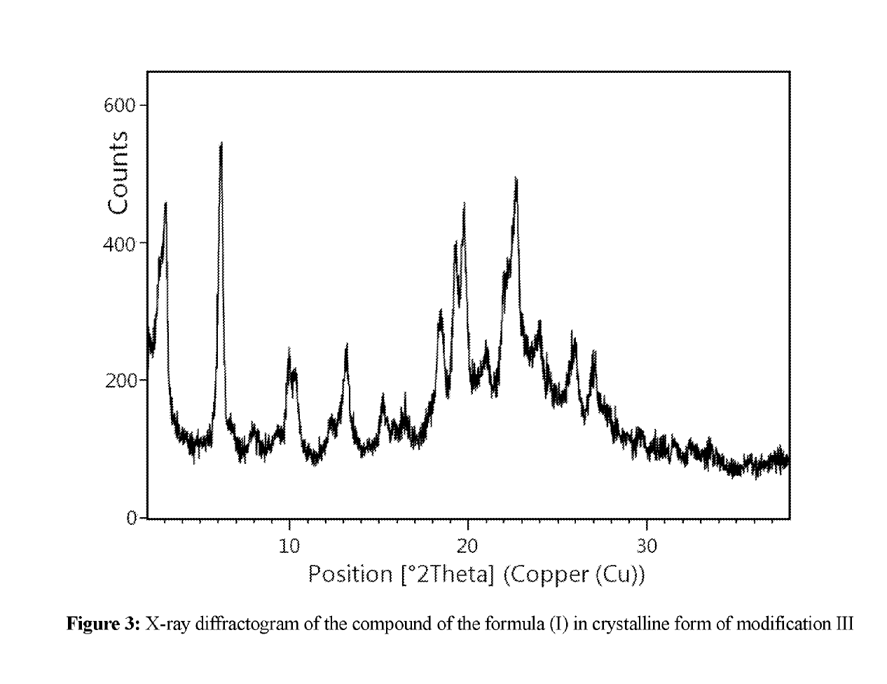

FIG. 4: X-ray diffractogram of the compound of the formula (I) in crystalline form of modification IV

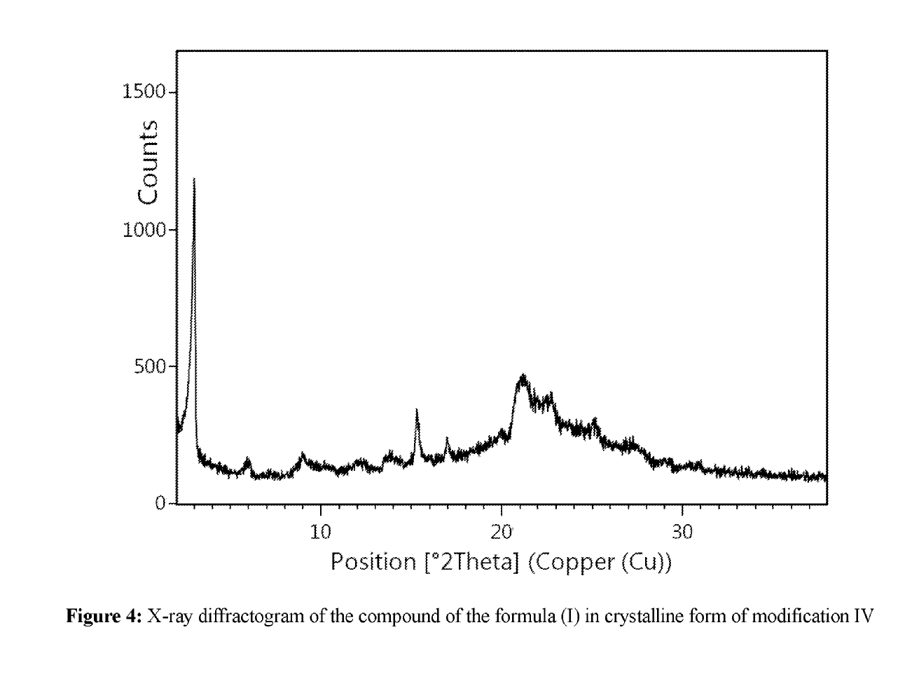

FIG. 5: X-ray diffractogram of the compound of the formula (I) in crystalline form of modification IV

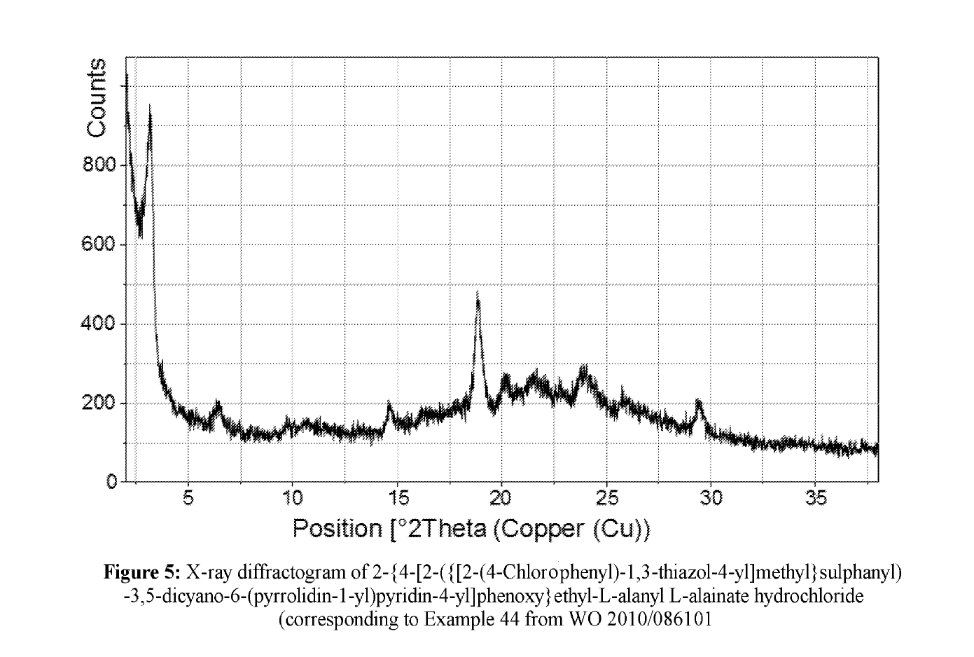

FIG. 6: Raman spectrum of compound of the formula (I) in crystalline form of modification I

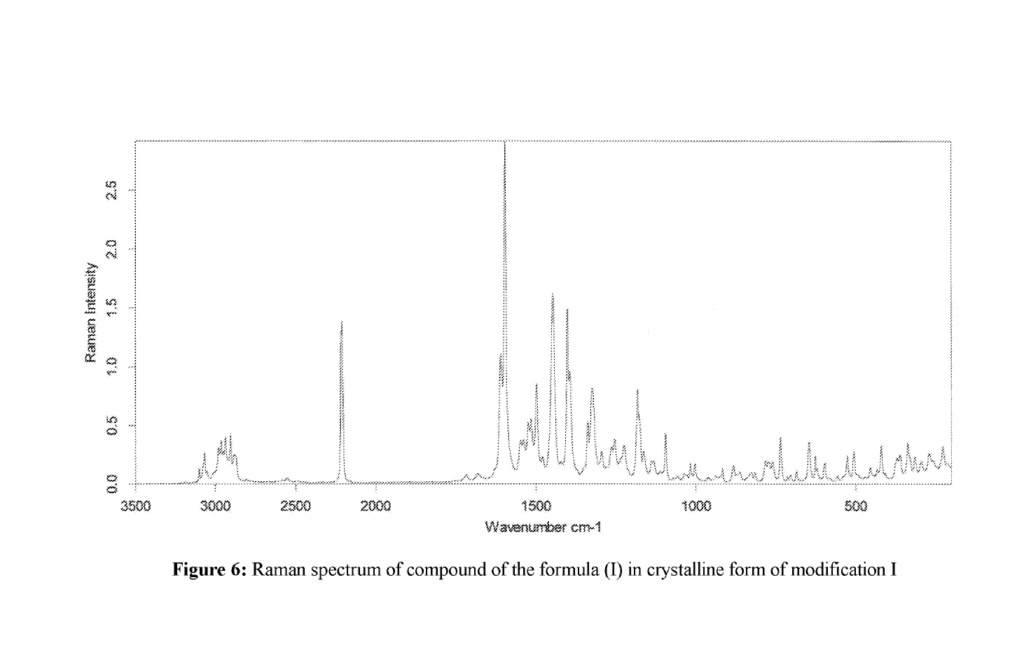

FIG. 7: Raman spectrum of compound of the formula (I) in crystalline form of modification II

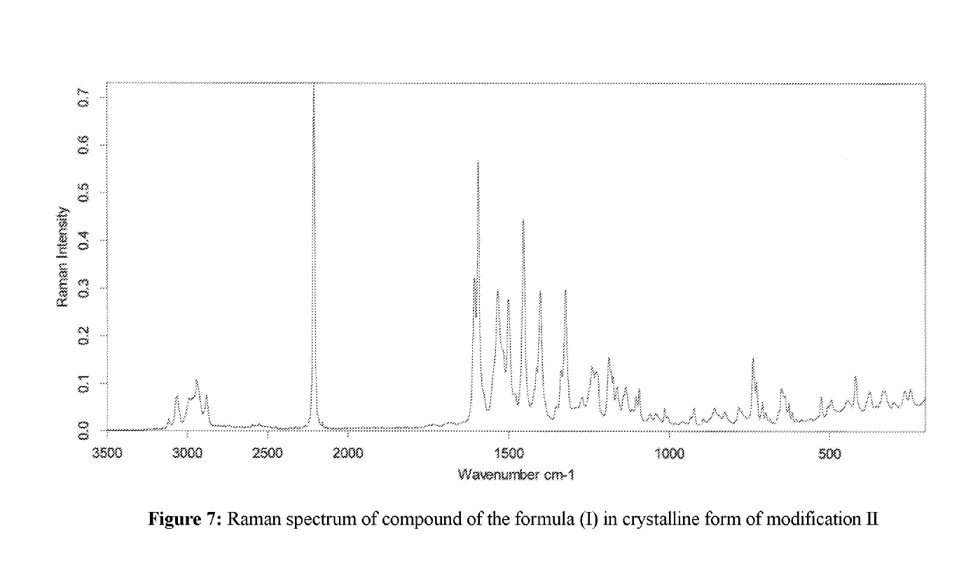

FIG. 8: Raman spectrum of compound of the formula (I) in crystalline form of modification III

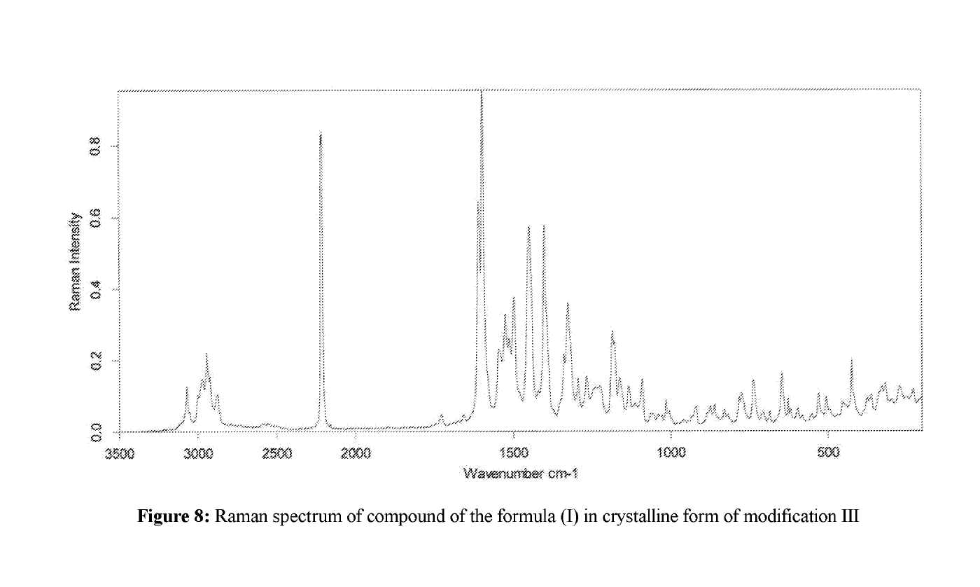

FIG. 9: Raman spectrum of compound of the formula (I) in crystalline form of modification IV

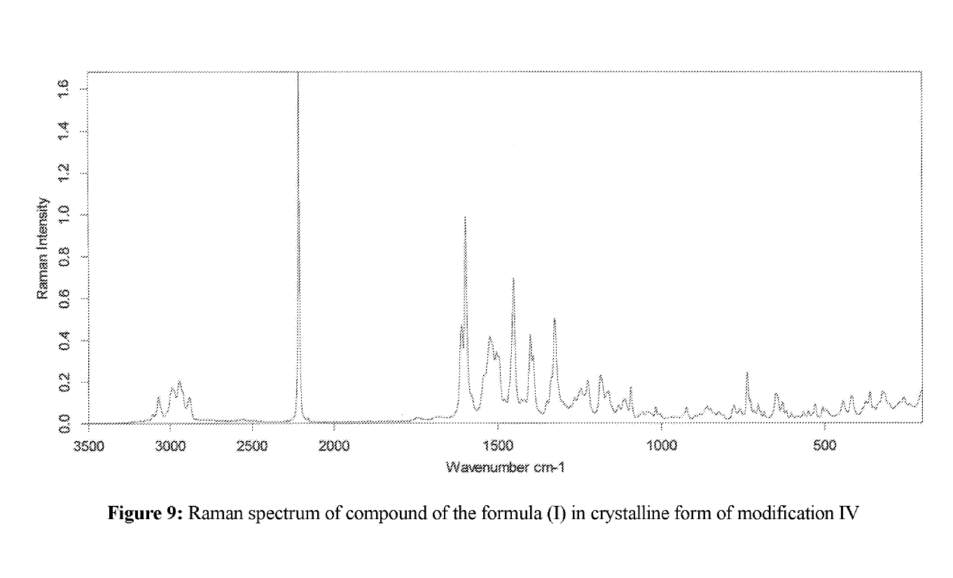

FIG. 10: Raman spectrum of compound of the formula (I) in crystalline form of the isopropanol solvate

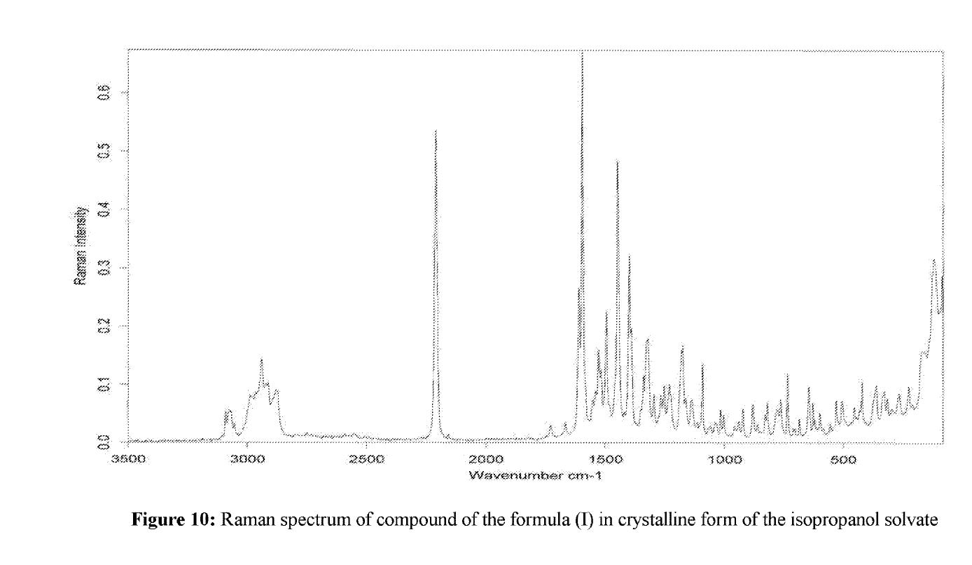

1. 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate monohydrochloride of the formula (I) in crystalline form of modification I 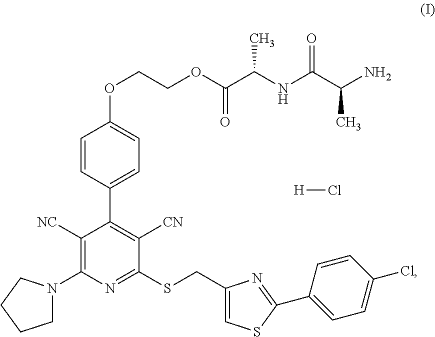 [SMILES: C[C@H](N)C(=O)N[C@@H](C)C(=O)OCCOc1ccc(-c2c(C#N)c(SCc3csc(-c4ccc(Cl)cc4)n3)nc(N3CCCC3)c2C#N)cc1 ; Cl]
wherein the X-ray diffractogram of the compound has peak maxima of the 2 theta angle at 6.5, 8.7 and 24.3.

2. Compound according to claim 1, wherein the X-ray diffractogram of the compound has peak maxima of the 2 theta angle at 6.5, 8.7, 18.3, 19.9, 20.7, 23.5 and 24.3.

3. 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate monohydrochloride of the formula (I) in crystalline form of modification I 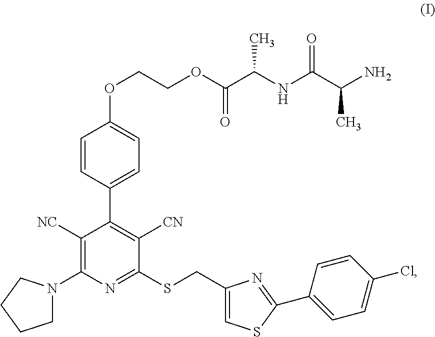 [SMILES: C[C@H](N)C(=O)N[C@@H](C)C(=O)OCCOc1ccc(-c2c(C#N)c(SCc3csc(-c4ccc(Cl)cc4)n3)nc(N3CCCC3)c2C#N)cc1]
wherein the Raman spectrum of the compound has band maxima at 2907, 1004 and 598 cm⁻¹.

4. Compound according to claim 3, wherein the Raman spectrum of the compound has band maxima at 2907, 1539, 1515, 1182, 1004 and 598 cm⁻¹.

5. Compound according to claim 3, wherein the Raman spectrum of the compound has band maxima at 2907, 1539, 1515, 1394, 1245, 1182, 1004 and 598 cm⁻¹.

6. Medicament comprising a compound according to claim 1 and no greater proportions of any other form of the compound of the formula (I).

7. Medicament comprising a compound according to claim 1 in more than 90 percent by weight based on the total amount of the compound of the formula (I) present.

8. Use of the compound according to claim 1 for production of a medicament for treatment of cardiovascular disorders or of kidney disorders.

9. Method for treatment of cardiovascular disorders or kidney disorders by administering an effective amount of a compound according to claim 1.

10. Process for preparing 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate monohydrochloride of the formula (I) in crystalline form of modification I, wherein the dialaninate dihydrochloride of the formula (IX) 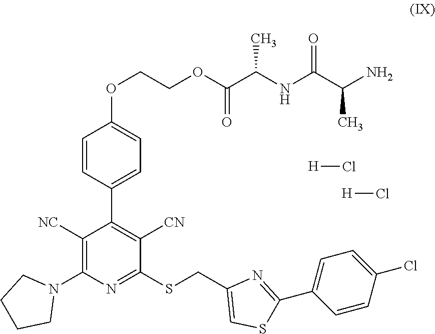
is stirred in an excess of isopropanol or n-propanol/water mixture (98:2) at room temperature, giving, after drying, the compound of the formula (I) in crystalline modification I.

11. Process according to claim 10 for preparing compound (I) 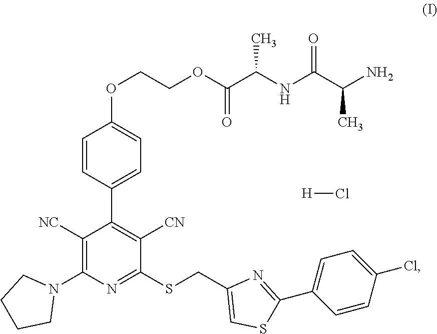 [SMILES: C[C@H](N)C(=O)N[C@@H](C)C(=O)OCCOc1ccc(-c2c(C#N)c(SCc3csc(-c4ccc(Cl)cc4)n3)nc(N3CCCC3)c2C#N)cc1 ; Cl]
wherein
[A] in step 1 in a one-pot reaction the compound of the formula (II) 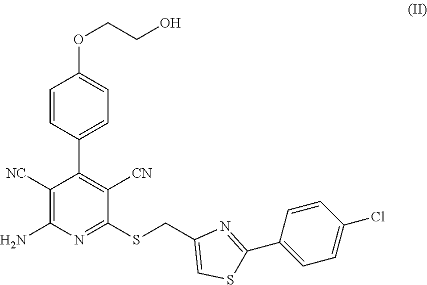 [SMILES: N#Cc1c(N)nc(SCc2csc(-c3ccc(Cl)cc3)n2)c(C#N)c1-c1ccc(OCCO)cc1]
is reacted in the presence of a solvent, a phase transfer catalyst, a nitrite and copper(II) chloride and the intermediate of the formula (III) obtained 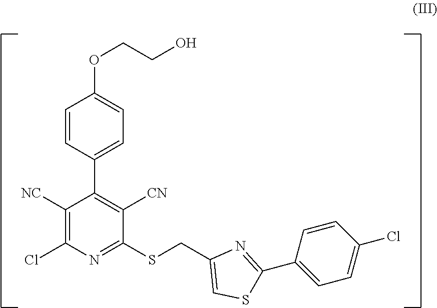
is reacted without isolation, i.e. in solution, with pyrrolidine to give the compound of the formula (IV) 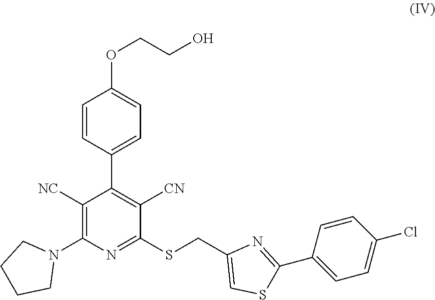 [SMILES: N#Cc1c(SCc2csc(-c3ccc(Cl)cc3)n2)nc(N2CCCC2)c(C#N)c1-c1ccc(OCCO)cc1]
and
[B] the compound of the formula (IV) obtained in this manner is, in a step 2, converted in a one-pot reaction with Boc-L-alanine in the presence of the condensing agent dicyclohexylcarbodiimide (DCC) in combination with 4-(dimethylamino)pyridine and in the presence of a solvent into the protected Boc-alaninate (V) 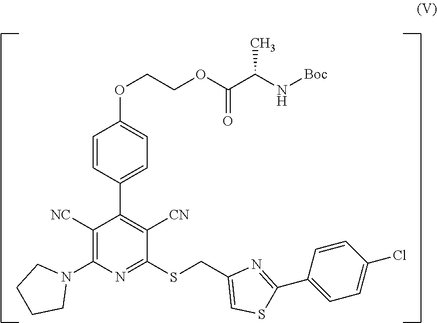
which is, without isolation, i.e. in solution, converted with hydrochloric acid into the alaninate dihydrochloride of the formula (VIII) 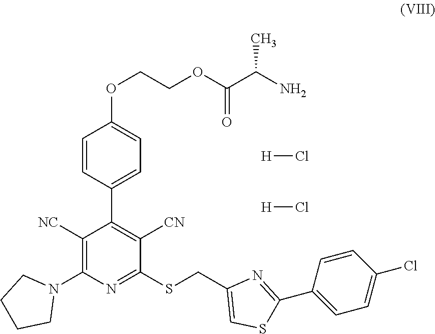 [SMILES: C[C@H](N)C(=O)OCCOc1ccc(-c2c(C#N)c(SCc3csc(-c4ccc(Cl)cc4)n3)nc(N3CCCC3)c2C#N)cc1 ; Cl ; Cl]
and
[C] the compound of the formula (VIII) obtained in this manner is, in a step 3, in combination with 4-(dimethylamino)pyridine and in the presence of a solvent, reacted with Boc-L-alanine in the presence of the condensing agent dicyclohexylcarbodiimide (DCC) to give the protected Boc-dialaninate (VII) 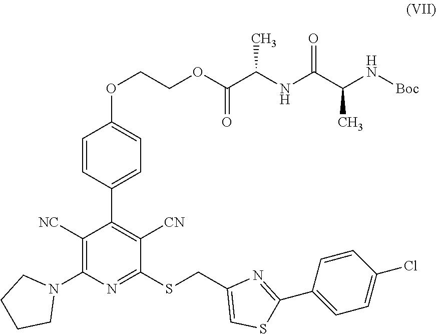 [SMILES: C[C@H](NC(=O)OC(C)(C)C)C(=O)N[C@@H](C)C(=O)OCCOc1ccc(-c2c(C#N)c(SCc3csc(-c4ccc(Cl)cc4)n3)nc(N3CCCC3)c2C#N)cc1]
and
[D] the compound of the formula (VII) obtained in this manner is, in a step 4, in the presence of a solvent, reacted with hydrochloric acid to give the dialaninate dihydrochloride of the formula (IX) 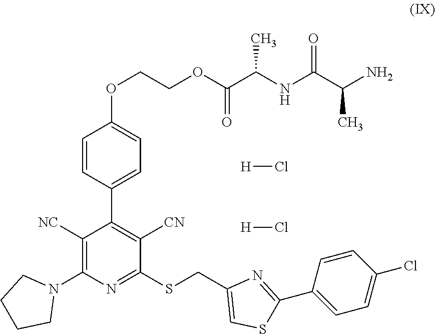 [SMILES: C[C@H](N)C(=O)N[C@@H](C)C(=O)OCCOc1ccc(-c2c(C#N)c(SCc3csc(-c4ccc(Cl)cc4)n3)nc(N3CCCC3)c2C#N)cc1 ; Cl ; Cl]
and
[E] the compound of the formula (IX) obtained in this manner is, in a step 5, stirred in an excess of isopropanol or n-propanol/water mixture (98:2) at room temperature, giving the compound of the formula (I) in crystalline modification I.

12. Process according to claim 11, wherein the compound of the formula (II) 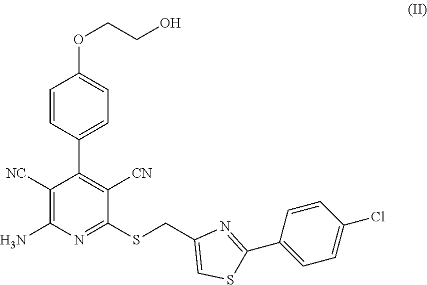 [SMILES: N#Cc1c(N)nc(SCc2csc(-c3ccc(Cl)cc3)n2)c(C#N)c1-c1ccc(OCCO)cc1]
is obtained by first, in the presence of methanol and triethylamine, reacting the substituted malononitrile of the formula (XI) 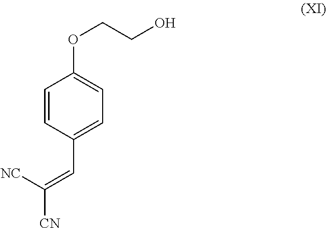 [SMILES: N#CC(C#N)=Cc1ccc(OCCO)cc1]
with cyanothioacetamide of the formula (XII) 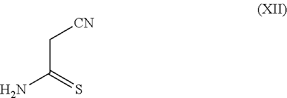
and then reacting the intermediate obtained with chloromethylchlorephenylthiazole of the formula (XIV)

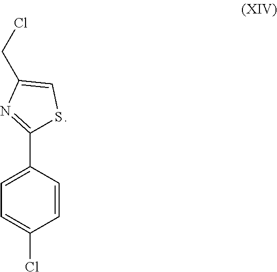 [SMILES: ClCc1csc(-c2ccc(Cl)cc2)n1]

13. Process according to claim 11, wherein in step 1 the intermediate of the formula (III) is isolated, that is obtained as a solid, prior to being reacted further.

14. Process according to claim 11, wherein in step 2 the protected Boc-alaninate of the formula (V) is isolated, that is obtained as a solid, prior to being reacted further.

15. Process according to claim 11, wherein the protected Boc-dialaninate of the formula (VII) obtained in step 3 is not isolated, but directly reacted further.

16. Process according to claim 11, wherein in step 1 the intermediate of the formula (III) is isolated, that is obtained as a solid, prior to being reacted further.

17. Compound 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate dihydrochloride of the formula (IX)

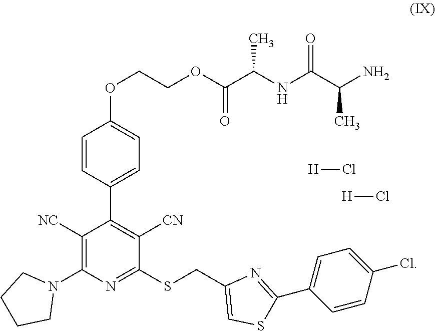

18. Compound 2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl L-alaninate dihydrochloride (VIII) [chemical structure]
